(19) United States
(12) Patent Application Publication
(10) Pub. No.: US 2026/0124262 A1
(43) Pub. Date: May 7, 2026

(54) COMPOSITIONS AND METHODS FOR THE TREATMENT OF DISEASES OR DISORDERS IN MAMMALIAN ANIMALS

(71) Applicant: GEROSYNTH LABORATORIES, INC., Bloomington, MN (US)

(72) Inventor: ALISHA S. O'DELL, Lakeville, MN (US)

(73) Assignee: GEROSYNTH LABORATORIES, INC., Bloomington, MN (US)

(21) Appl. No.: 19/378,893

(22) Filed: Nov. 4, 2025

Related U.S. Application Data
(60) Provisional application No. 63/716,808, filed on Nov. 6, 2024.
(60) Provisional application No. 63/716,811, filed on Nov. 6, 2024.

(51) Int. Cl. A61K 36/07; A61K 9/107; A61P 35/00; A61P 37/08

(52) U.S. Cl. CPC A61K 36/07; A61K 9/107; A61P 35/00; A61P 37/08

(57) ABSTRACT

Pharmaceutical compositions, and methods of production and use for the treatment of neoplastic disease in a mammalian animal. When administered to an animal, the pharmaceutical compositions are useful for the treatment of neoplastic diseases such as various cancers, particularly those affecting mammalian animals such as companion animals.

### INCORPORATION BY REFERENCE

Applicant's pending PCT/US24/27210, filed May 1, 2024 is incorporated by reference herein in its entirety.

### PRIORITY

The present application claims the full benefit and priority of U.S. provisional applications 63/716,808 and 63/716,811, both filed Nov. 6, 2024. They are likewise fully incorporated by reference.

### FIELD

The present invention relates generally to pharmaceutical compositions and methods for the treatment of a dermatologic, neurologic or neoplastic disease in mammalian animals.

### BACKGROUND OF THE INVENTION

Here follows a preliminary discussion of the invention, which is not necessarily prior art, and should not necessarily be so construed.

Dermatologic diseases in household pets are common. While all pets can have dermatologic diseases or disorders, they are most commonly observed in dogs. These diseases can arise from allergies, parasites, infections, or underlying internal issues. Examples include atopic dermatitis (environmental allergies), flea allergy dermatitis, contact dermatitis, bacterial and fungal infections, such as pyoderma and ringworm, and parasitic infestations such as mites and scabies. Symptoms frequently include itching, hair loss, skin redness, and secondary skin infections, requiring veterinary diagnosis, skin baths and ointment treatments, injections, administration of pharmaceuticals, and other treatment tailored to the specific cause. Atopic dermatitis is an inflammatory skin disease caused by a reaction to environmental allergens such as pollen, mold and dust mites. Flea allergy dermatitis is an allergic reaction to flea bites, causing intense itching (pruritus), skin irritation and possible hair loss (alopecia). Bacterial infections are often secondary to other conditions, the most common of which is pyoderma, an infection of hair follicles. Fungal infections, such as ringworm (dermatophytosis), a highly contagious skin infection, can spread from one animal to another, possibly including humans. Other fungal infections include systemic diseases such as blastomycosis, histoplasmosis and cryptococcosis. Parasitic infestations such as fleas, ticks, and mites, can cause itching and transmit diseases. Autoimmune diseases and disorders can also cause dermatologic problems as well. For example, pemphigus is an autoimmune disease in which the body's immune system attacks its own skin cells. In addition, hormonal disorders can cause skin issues, hair loss or changes in skin quality.

Common neurologic diseases in pets can cause seizures, difficulty walking, and head tilting, and include conditions such as epilepsy, stroke, brain tumors, vestibular disease, degenerative myelopathy, and some infectious diseases. Symptoms vary by condition but may also include changes in behavior, loss of coordination, tremors, and paralysis. Diagnosis requires a veterinary exam and diagnostic tests, with treatments ranging from medication to surgery.

Cancer is a disease that impacts pet owners around the world and is the leading cause of death in pets middle-aged and older. The increase in cancer diagnosis rates for dogs in the past 10 years has skyrocketed. According to the Veterinary Cancer Society, an average of one in four dogs will be diagnosed with cancer, with very limited treatment options that only promise to extend the life of the dog for a short time.

The long-term prognosis for the survival of dogs with cancer is grim. And often the costs of treatment are prohibitive.

Pet owners around the world are in desperate need of a real solution that delivers lasting results. There are no long-term treatment options for equines having advanced-stage cancer.

Therefore, there is a critical need for novel, effective and less toxic therapeutic approaches to cancer, dermatologic and neurologic treatments for canine, feline, equine and other mammalian animals.

Medicinal mushrooms and other natural products have been used to treat various diseases and infections for hundreds of years around the world. Today, medicinal mushrooms are used to treat many types of diseases including lung diseases and many types of cancer around the world. For more than 30 years, medicinal mushrooms have been approved as an addition to standard cancer treatments in Japan and China. In these countries, mushrooms have been used safely for a long time, either alone or combined with radiation or various chemotherapeutics. More than 100 different types of mushrooms are used to treat cancer in Asia, including Ganoderma lucidum (relish), Trametes versicolor or Curious versicolor (turkey tail), Lentinus edodes (shiitake), and Grifola frondosa (maitake). Since many mushrooms are edible and non-toxic, they provide the possibility that one or more of their constituent compounds may provide an effective, non-toxic treatment for cancer.

Of particular interest is how mushrooms affect the immune system and if they stop or slow the growth of tumors or kill tumor cells. It is thought that certain chemical compounds, such as polysaccharides (beta-glucans), triterpenes, alkaloids, and other forms of biomolecules present in mushrooms strengthen the immune system to fight cancer directly as well as exert direct anticancer functions on the cancer cells themselves. Potential uses of mushrooms, both individually and as adjuncts to cancer therapy have emerged. Mushrooms are also known to complement chemotherapy and radiation therapy by countering the side-effects of cancer, such as nausea, bone marrow suppression, anemia, and lowered resistance. Recently, a number of bio-active molecules, including anti-tumor agents, have been identified from various mushrooms. These bioactive molecules include polysaccharides, proteins, fats, ash, glycosides, alkaloids, volatile oils, tocopherols, phenolics, flavonoids, carotenoids, folates, ascorbic acid enzymes and organic acids. Polysaccharides, such as Beta-D-glucan, are the widest known mushroom-derived compounds with anti-cancer and immunomodulating properties. For example, polysaccharide K (PSK), a protein-bound polysaccharide found in turkey tail mushrooms, is an approved mushroom product used to treat cancer in Japan and has been studied as an adjuvant therapy in the treatment of gastric (stomach) cancer, breast cancer, colorectal cancer and lung cancer.

A dietary supplement prepared from Trametes versicolor; the turkey tail mushroom, has been shown to reduce the growth of hormone-responsive prostate cancer LNCaP cells. A crude extract of T. versicolor has been shown to inhibit growth in a number of human cancer cell lines, including gastric cancer (7907), lung cancer (SPC), leukemia (MCL) and lymphoma (SLY). In addition, a polysaccharide of this mushroom has been shown to inhibit the proliferation of human hepatoma cancer (QCY) cells in vitro and in vivo, which occurs with apoptosis and a decrease in the expression of the cell cycle-related genes, p53, Bcl-2 and Fas.

The mushroom genus Ganoderma, commonly known as Reishi or Lingzhi, has traditionally been administered throughout Asia as an anti-cancer agent for centuries. Extracts of G. lucidur have been shown to decrease the viability of human gastric carcinoma cells. Ganoderic acid T (GA-T) has been shown to inhibit tumor invasion and metastasis in human colon cancer cells lines, while other ganoderic acids, including (GA-Me, GA-Mf, GA-S) have been shown to be cytotoxic to human colon carcinoma cells and to decrease cell population growth in human carcinoma cell lines. A native glycopeptide, LZ-D-4, purified from the fruiting bodies of G. lucidur and its sulfated derivative, LZ-D, exhibited anti-tumor activity in vitro against mouse lymphocytic leukemia.

A d-glucan purified from Grifola frondosa, known as the dancing mushroom or Maitake, has been shown to enhance the efficacy of cisplatin, checking the decrease in the number of immunocompetent cells, namely macrophages, DCs and NK cells in cisplatin-treated mice. A chemically-sulfated polysaccharide (S-GAP-P) derived from a water insoluble polysaccharide of G. frondosa, has been shown to have anti-cancer effects when used in combination with 5-fluorouracil (5-FU) in human carcinoma cells, inhibiting cell growth and inducing cell apoptosis. A polysaccharide-peptide, GFPPS1b, isolated from cultured mycelia of G. frondosa was shown to have anti-tumor activity which inhibited the proliferation of human gastric adenocarcinoma cells. The cells succumbed to apoptosis, which was associated with a drop in mitochondrial transmembrane potential, up-regulation of Bax, down regulation of Bsl-2, and activation of caspase-3.

Several different additional mushroom types have been the subject of anti-cancer studies. A water and ethanol extract of one of these, the Chaga mushroom (Inonotus obliquus), has been shown to induce apoptosis in human colon cancer (DLD-1) cells by prevention of reactive oxygen species (ROS)-induced tissue damage, among other functions. A water extract of Chaga was also shown to arrest the cell cycle at the Go/G1 phase in B16-F10 murine melanoma cells, causing not only apoptosis, but also induced cell differentiation. These effects were associated with the down-regulation of pRb, p53 and p27 expression levels, and further shows that the Chaga extract resulted in a Go/G₁ cell cycle arrest with reduction of cyclin E/D1 and Cdk 2/4 expression levels. Furthermore, the anti-tumor effect of Chaga extract was assessed in vivo in Balb/c mice. Intraperitoneal administration of Chaga extract significantly inhibited the growth of tumor mass in B16-F10 cells implanted in mice, resulting in 3-fold inhibition at a dose of 20 mg/kg/day for 10 days. The ethanolic extract of sclerotium and fruiting body of Chaga elicited significant anti-tumor activity 74.6% and 44.2% respectively.

The pentacyclic triterpenoid betulinic acid occurs naturally in Chaga and has been the subject of a number of studies for its anti-cancer properties due to its anti-tumoral activity and the ability to overcome resistance by inducing apoptosis in a variety of human cancers. Its selective cytotoxicity against cancer was first described in human melanoma both in vitro and in vivo in 1995. Since then, betulinic acid has been reported to be effective on a number of human cancers, including cancers of the lung, colon, prostate, and ovary. One study has shown that normal cells remain unaffected by betulinic acid treatment. Betulinic acid has also been applied in vitro in childhood cancers, viz medulloblastoma, glioblastoma, Ewing sarcoma, neuroblastoma, and leukemia. Accumulated experimental evidence shows that betulinic acid treatment results in morphological change in sensitive cells, such as cell shrinkage, DNA fragmentation, nuclear condensation, and membrane blebbing. While the exact molecular mechanism underlying betulinic acid-induced apoptosis remains unclear, several studies suggest that the proteolytic cleavage of caspases, the activation of the MAP kinase cascade, the modulation of NF-KB signaling, the generation of reactive oxygen species, and the inhibition of topoisomerase I may all be contributing processes. Along with these functions, betulinic acid has also been found to reactivate the mitochondria, and induce apoptosis by releasing cytochrome C, a signaling protein that induces apoptosis naturally in response to irreparable cellular damage.

There is a need to further explore the potential benefits of Chaga mushrooms, particularly the potential oncological benefits. One limitation discovered in the continuing research of betulinic acid, and discussed openly among many research groups, is the poor bioavailability of betulinic acid. Scientists have speculated that this low bioavailability is caused by the poor water solubility of betulinic acid. The low bioavailability of betulinic severely limits practical applications of betulinic acid as a therapeutic agent. Various attempts to overcome this low bioavailability have been tried and have included self-nanoemulsifying drug delivery systems and spray gun technologies, but none have provided a practical solution. Therefore, what is needed is a betulinic acid or derivative thereof having sufficient bioavailability to provide a therapeutic effect when administered to humans and animals, particularly mammalian animals.

### SUMMARY

Methods of treating mammalian diseases and disorders are provided herein. The diseases and disorders to be treated include dermatologic diseases, neurologic diseases and neoplastic diseases, or cancers, particularly in mammals such as canines, felines and equines.

Examples of dermatologic diseases to be treated with the methods described herein include atopic dermatitis, flea allergy dermatitis, contact dermatitis, bacterial and fungal infections, such as pyoderma and ringworm, and parasitic infestations such as mites and scabies. Common symptoms are itching, hair loss, skin redness, and secondary skin infections.

Examples of neurologic diseases to be treated with the methods described herein are epilepsy, stroke, brain tumors, vestibular disease, degenerative myelopathy, some infectious diseases, traumatic brain injury, trauma to the central nervous system, trauma to the peripheral nervous system, drug trauma, traumatic accidental injury (such as being hit by a car), and blunt force trauma. Symptoms vary by condition but may also include changes in behavior, loss of coordination, loss of movement, tremors, and paralysis.

A neoplastic disease is a condition caused by an abnormal, uncontrolled growth of cells, referred to as a neoplasm or tumor. Neoplasms can be benign, in that they are localized and do not spread, or malignant, which are cancerous and can invade surrounding tissue or metastasize. Cancer is a leading cause of death of pets and other domestic mammals.

In accordance with the methods provided herein, a therapeutic dose of a Medicinal Formulation, as defined herein, is administered to a mammalian animal exhibiting symptoms of, or diagnosed with, a dermatologic, neurologic or neoplastic disease. The Medicinal Formulation is ideally combined with a carrier to create a composition that facilitates administration, particularly oral administration, to animals such as pets. A suitable therapeutic dose for oral administration is from 0.5 ml/10 lbs. of body weight to 3.0 ml/10 lbs. of body weight.

The Medicinal Formulation is an emulsified Chaga mushroom solution containing chemical-modified compounds of the Chaga mushroom produced by combining a reducing sugar with an amino acid and heated the combination at an elevated temperature under vacuum for a sufficient amount of time to form one or more Maillard reaction products. The one or more Maillard reaction products are added to Chaga mushrooms, and the solution is brought to a boil under vacuum. The Chaga mushroom reaction product is esterified with a fatty acid under similar reaction conditions to produce an emulsion or micelles. The emulsion or micelles contain chemically-modified compounds of the Chaga mushroom.

In one embodiment the Medicinal Formulation contains a chemical compound having the formula of Formula 1:

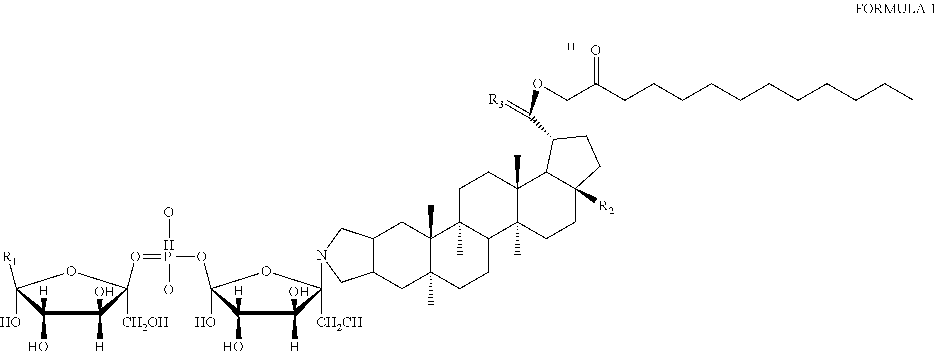

wherein R¹, R² and R³ are independently selected from the group consisting of alkyl/alkane, alkene/alkenyl, or alkyne/alkynyl, having from 1 to 20 carbon atom; benzene/aromatic/phenyl, ether, amide, alkyl halide, amine (-amino), alcohol/hydroxy/hydroxyl (—OH), thiol, aldehyde, ketone, ester, carboxylic acid (COOH), acid anhydride, acyl halide, or methyl; or wherein at least one of R¹, R² and R³ is a hydroxyl group; or wherein R¹, R² and R³ are hydroxyl groups.

In another embodiment, the Medicinal Formulation contains a chemical compound having the formula of Formula 2:

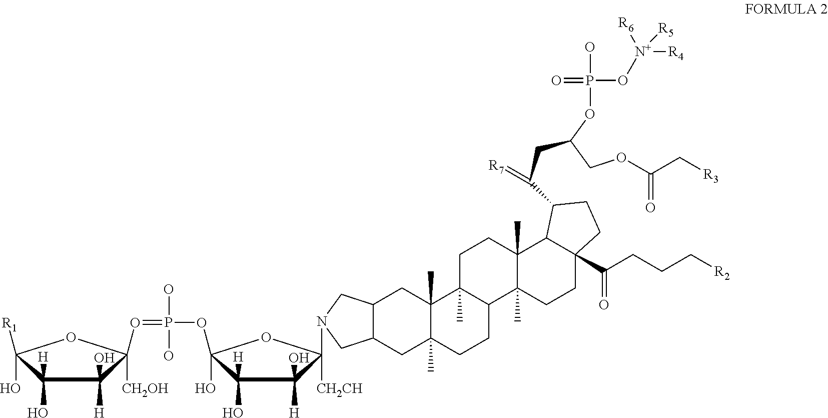

wherein R₁-R₇ are, independently, alkyl/alkane, alkene/alkenyl, or alkyne/alkynyl, having from 1 to 20 carbon atom; benzene/aromatic/phenyl, ether, amide, alkyl halide, amine (-amino), alcohol/hydroxy/hydroxyl (—OH), thiol, aldehyde, ketone, ester, carboxylic acid (COOH), acid anhydride, acyl halide, or methyl; or wherein R₁-R₇ are, independently, alkyl/alkane, alkene/alkenyl, or alkyne/alkynyl, having from 1 to 20 carbon atom; benzene/aromatic/phenyl, ether, amide, alkyl halide, amine (-amino), alcohol/hydroxy/hydroxyl (—OH), thiol, aldehyde, ketone, ester, carboxylic acid (COOH), acid anhydride, acyl halide, or methyl, and wherein at least one of R₁-R₇ is a hydroxyl group; or wherein R₁-R₇ are hydroxyl groups.

The following are additional various concepts numbered for reference in other concepts.
Concept 1—A method of treating a neoplastic disease in a mammalian subject, by administering to the subject an effective amount of a Medicinal Formulation, wherein the Medicinal Formulation is an emulsified Chaga mushroom solution containing chemical-modified compounds of the Chaga mushroom.
Concept 2—The method of Concept 1, wherein the neoplastic disease is cancer.
Concept 3—The method of Concept 1, wherein the cancer is melanoma, carcinoma, sarcoids, prostate cancer, colorectal cancer, breast cancer, liver cancer and brain cancer.
Concept 4—The method of Concept 1, wherein the carcinoma is squamous cell carcinoma, basal cell carcinoma, or Merkel-cell carcinoma
Concept 5—A method of treating a dermatologic disease in a mammalian subject, by administering to the subject an effective amount of a Medicinal Formulation, wherein the Medicinal Formulation is an emulsified Chaga mushroom solution containing chemical-modified compounds of the Chaga mushroom.
Concept 6—The method of Concept 5, wherein the dermatologic disease is atopic dermatitis, flea allergy dermatitis, contact dermatitis, bacterial infections, fungal infections or parasitic infestation.
Concept 7—The method of Concept 6, wherein the dermatologic disease is a bacterial infection, and the bacterial infection is pyoderma
Concept 8—The method of Concept 6, wherein the dermatologic disease is a fungal infection, and the fungal infection is ringworm
Concept 9—The method of Concept 6, wherein the dermatologic disease is a parasitic infestation and the parasitic infection is selected from the group consisting of mites and scabies
Concept 10—A method of treating a neurologic disease in a mammalian subject, by administering to the subject an effective amount of a Medicinal Formulation, wherein the Medicinal Formulation is an emulsified Chaga mushroom solution containing chemical-modified compounds of the Chaga mushroom
Concept 11—The method of Concept 10, wherein the neurologic disease is epilepsy, stroke, a brain tumor, a vestibular disease, degenerative myelopathy, an infectious disease or a trauma
Concept 12—The method of Concept 11 wherein the trauma is traumatic brain injury, trauma to the central nervous system, trauma to the peripheral nervous system, drug trauma, traumatic accidental injury, or blunt force trauma
Concept 13—The method of Concept 1, 5 or 10 wherein the effective amount is a therapeutic dose of the Medicinal Formulation that treats the disease
Concept 14—The method of Concept 13, wherein the therapeutic dose is from 0.5 mi/10 lbs. of body weight to 3.0 ml/10 lbs. of body weight
Concept 15—The method of Concept 1, 5 or 10, wherein the administration is oral administration
Concept 16—The method of Concept 15, wherein, the Medicinal Formulation is combined with a carrier to create a composition that facilitates oral administration
Concept 17—The method of Concept 1, 5 or 10, wherein the Medicinal Formulation contains a chemical compound having the formula of Formula 1, wherein R¹, R² and R³ are independently selected from the group consisting of alkyl/alkane, alkene/alkenyl, or alkyne/alkynyl, having from 1 to 20 carbon atom; benzene/aromatic/phenyl, ether, amide, alkyl halide, amine (-amino), alcohol/hydroxy/hydroxyl (—OH), thiol, aldehyde, ketone, ester, carboxylic acid (COOH), acid anhydride, acyl halide, or methyl, in a pharmaceutically-acceptable carrier; or wherein at least one of R¹, R² and R³ is a hydroxyl group; or wherein R¹, R² and R³ are hydroxyl groups
Concept 18—The method of Concept 1, 5 or 10, wherein the Medicinal Formulation contains a chemical compound having the formula of Formula 2, wherein R₁-R₇ are, independently, alkyl/alkane, alkene/alkenyl, or alkyne/alkynyl, having from 1 to 20 carbon atom; benzene/aromatic/phenyl, ether, amide, alkyl halide, amine (-amino), alcohol/hydroxy/hydroxyl (—OH), thiol, aldehyde, ketone, ester, carboxylic acid (COOH), acid anhydride, acyl halide, or methyl; or wherein R₁-R₇ are, independently, alkyl/alkane, alkene/alkenyl, or alkyne/alkynyl, having from 1 to 20 carbon atom; benzene/aromatic/phenyl, ether, amide, alkyl halide, amine (-amino), alcohol/hydroxy/hydroxyl (—OH), thiol, aldehyde, ketone, ester, carboxylic acid (COOH), acid anhydride, acyl halide, or methyl, and wherein at least one of R₁-R₇ is a hydroxyl group; or wherein R₁-R7 are hydroxyl groups.

### BRIEF DESCRIPTION OF THE DRAWINGS

The invention may be more completely understood in consideration of the following detailed description of various embodiments of the invention in connection with the accompanying drawings, in which:

FIGS. 1-7 show the results of in vivo testing to demonstrate the chemotherapeutic effects of the pharmaceutical composition on a canine tumor. The in vivo tests were performed on a canine subject (beagle dog) suffering from osteosarcoma/mast cell/round cell on the hind leg. The cytotoxic effects, apoptosis-inducing effects, and reduction in tumor size were observed.

FIG. 1 is a photograph of a microscope slide showing the initiation of apoptosis in cancer cells from a biopsy sample from a leg tumor of a canine subject undergoing treatment with the pharmaceutical composition described herein.

FIG. 2 is a photograph of a microscope slide showing the initiation of apoptosis in cancer cells from a biopsy sample from a leg tumor of a canine subject undergoing treatment with the pharmaceutical composition described herein at higher resolution.

FIG. 3 is a photograph of a microscope slide showing cancer cells being engulfed by a macrophage from a biopsy sample from a leg tumor of a canine subject undergoing treatment with the pharmaceutical composition described herein.

FIG. 4 is photograph of a microscope slide showing cancer cells being destroyed by mast cells/neutrophils from a biopsy sample from a leg tumor of a canine subject undergoing treatment with the pharmaceutical composition described herein.

FIG. 5 is a photograph of a microscope slide showing cancer cells being further destroyed by mast cells/neutrophils from a biopsy sample from a leg tumor of a canine subject undergoing treatment with the pharmaceutical composition described herein.

FIG. 6 is a photograph of a microscope slide showing cancer cells being further destroyed by mast cells/neutrophils from a biopsy sample from a leg tumor of a canine subject undergoing treatment with the pharmaceutical composition described herein, at higher resolution.

FIG. 7 is a photograph of a microscope slide showing cancer cells being further destroyed and aggregated by mast/neutrophils cells from a biopsy sample from a leg tumor of a canine subject undergoing treatment with the pharmaceutical composition described herein.

FIG. 8 is a photograph of a microscope slide showing cancer cells being further destroyed and aggregated by mast cells/neutrophils from a biopsy sample from a leg tumor of a canine subject undergoing treatment with the pharmaceutical composition described herein, at higher resolution.

FIG. 9 is a photograph of a microscope slide showing widespread destruction and aggregation of cancer cells having undergone apoptosis and engulfment by mast cell/neutrophils from a biopsy sample from a leg tumor of a canine subject undergoing treatment with the pharmaceutical composition described herein.

FIG. 10 is a photograph of rear view of a canine tumor showing the size of the tumor on the rear right leg of a canine patient in centimeters on day 0 immediately prior to the initiation of treatment.

FIG. 11 is a photograph of a lateral view of a canine tumor showing the size of the tumor on the rear right leg of a canine patient in centimeters 60 days after treatment was initiated.

FIG. 12 is a photograph of a lateral view of a canine tumor showing the size of the tumor on the rear right leg of a canine patient in centimeters, 120 days after treatment was initiated.

FIG. 13 is a photograph of a rear view of a canine tumor showing the size of the tumor in centimeters, 120 days after treatment was initiated.

FIG. 14 is a photograph of the shoulders and back of a dog suffering from atopic dermatitis on Day 01 of a study designed to determine the efficacy of administration of the Medicinal Formulation. Fur is very thin or absent.

FIG. 15 is photograph of the dog shown in FIG. 14 taken from the rear of the dog on Day 01 of the study. Patches of baldness and fur thinning can be seen.

FIG. 16 is a photograph of the dog shown in FIGS. 14 and 15 on Day 14 of treatment. Fur has grown in full and luxurious from head to tail.

FIG. 17 is a line graph of clinical symptoms for each of seven (7) dogs to whom have been administered the Medicinal Formulation. The line marked “A” represents the clinical symptom score on Day 0 of treatment, or oral administration of the Medicinal Formulation. The line marked “B” represents the clinical symptom score on Day 60 of treatment. And the line marked “C” represents the clinical symptom score on Day 90 of treatment.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION

Described herein are pharmaceutical compositions containing chemical compounds. Also described are methods of using the pharmaceutical compositions to treat neoplastic diseases such as cancers in animals.

Also described herein is a pharmaceutical composition containing a chemical compound wherein the chemical compound is isolated from a formulation or is synthesized and is combined with a pharmaceutically-acceptable carrier to form the pharmaceutical composition.

Also described herein is a method of treating cancer by administering one or more of the medicinal formulations, chemical compounds, or pharmaceutical compositions to an animal.

The chemical compounds in the pharmaceutical composition can be synthesized or isolated from a formulation as described in PCT/US2024/027210, filed May 1, 2024, which is incorporated by reference herein in its entirety. Briefly, the compounds are isolated by preparing a Chaga mushroom reduction in a reduction solvent under pressure, preparing a Chaga mushroom extraction in an extraction solvent, mixing or combining the Chaga reduction with the Chaga extraction, mixing or combining the Chaga formulation with an esterification mixture containing proline, fructose, and a fatty acid such as medium chain triglyceride (MCT) oil or sunflower lecithin, or other carbon chain fatty acids of various carbon chain length, with and without phospholipids for the development of esterified compounds, also including sources of sugar esters capable of emulsion within otherwise non-miscible liquid compounds, or a predetermined amount of Manuka honey, sufficient to cause esterification and facilitate emulsion at a temperature capable of inducing a reaction between the constituents of the Chaga mushroom with the constituents of the esterification mixture, such as Manuka honey, and combining that heterogenous mixture with a medium chain triglyceride.

The type of reaction most likely induced first is known as a Maillard reaction, also called a non-enzymatic browning reaction and is responsible for the formation of intermediates that would typically form a known end-product but are redirected into a novel reaction with betulinic acid and possibly other constituents found in the Chaga mushroom reductions and extracts to form novel compounds, as described herein. The Maillard reaction starts with a reaction between a reducing sugar and various types of amino acids when certain reaction parameter conditions are met. Two of the several intermediate products formed during the beginning of this reaction are known as Amadori and Heyns products. The full elucidation of a Maillard reaction remains unknown, but the various possible outcomes have been studied, as described herein, and measurements confirming the formation of these products have been achieved, as also described herein.

### Chaga Mushroom

The compound of greatest interest that can be extracted from the Chaga mushroom is betulinic acid (also referred to herein as “BA”). Betulinic acid is a pentacyclic triterpene, recently shown to have anti-cancer properties, but lacking in any clinical results. Betulinic acid on its own has limited bioavailability, having a molar mass of 456.7 g/mol and boiling point of 550° C. Past studies of betulinic acid have pointed to potential limiting factors in its usage, including its molecular size, poor aqueous solubility and low bioavailability. In its raw organic form in the Chaga mushroom, betulinic acid is bound to chitin, the primary component of cell walls in fungi. Extraction of betulinic acid from the Chaga mushroom, therefore, must include releasing the betulinic acid from its chitin binding.

Betulinic acid has been the subject of numerous studies and has been shown to induce apoptosis and to defragment DNA by inhibiting topoisomerase in the mitochondria of cancer cells. There has also been a suggestion that betulinic acid has an ability to reverse the Warburg effect, a form of modified metabolism found in cancer cells which favor a specialized fermentation of the aerobic respiration pathway preferred by most other cells of the body. In this fermentation process, the last product of glycolysis, pyruvate, is converted into lactate or ethanol, while yielding lower amounts of ATP than in the citric acid cycle. However, it allows cancer cells to convert glucose and glutamine into biomass by avoiding catabolic oxidation into carbon dioxide, thus preserving carbon-carbon bonds, and promoting anabolism. The mechanism by which betulinic acid can interrupt the Warburg effect is currently unknown, although the induction of cellular respiration and reversal of the fermentation process of glucose, the cancer cells' main pathway of utilizing glucose, could be a meaningful adjunct process for combatting the growth of cancer cells.

In addition to these potential functions, betulinic acid along with other bioactive compounds found in the Chaga mushroom are known to break down lactate at high rates. The removal of lactate can assist with combating the acidic, immune system-hostile, environment produced by cancer cells: the relatively high quantity of lactic acid creates a more acidic environment for the cancer cells, which favor a lower pH level than healthy cells. Some literature suggests that the lactic acid may also assist in providing for cancer cells to avoid detection by the immune system, allowing them to evade destruction by the immune cells. There are specific immune cells that when activated can detect and destroy cancer cells. Some of these types of cells are T-cells, specifically programmed to destroy cancer cells. When these immune cells can identify cancerous cells, they are quite capable of destroying the cancer cells.

It has also been suggested that betulinic acid may preferably create an oxidative stress load in cancer cells, and that this stress load may be a factor in how betulinic acid inhibits topoisomerase, and thus inhibiting DNA production, while seeming to be harmless to the surrounding healthy cells.

Also of interest is the high level of superoxide dismutase (SOD) in both Chaga extract and Chaga reduction. SOD's may be useful in dismutating the reactive oxygen species (ROSs) produced by the cancer cell's preferred mode of action and a part of the cellular damage cycle that induces signaling possibly linked to the spread of metastatic disease. Normal healthy cells produce their own SODs to manage oxidative stress from radical oxygen species to maintain cellular health, but cancer cells become unable to manage cellular damage and lose the ability to signal apoptosis due to cellular damage. The high content of SODs provided in the formulation may be of assistance to particular cancer cells in repairing damage and establishing a normal metabolic function once again.

There are potentially additional compounds present in Chaga that can benefit the host's immune cell's ability to counter-act cancer progression.

### Manuka Honey

Manuka honey is a monofloral or multifloral honey produced from nectar of the manuka plant (Leptospermum scoparium), which is native to south-east Australia and New Zealand.

A test for monofloral Manuka honey, adopted by the New Zealand Ministry for Primary Industries (see https://www.mpi.govt.nz/food-business/honey-bee-products-processing-requirements/manuka-honey-testing/) is that the honey has the five following characteristics:
contains 3-phenyllactic acid at a level greater than or equal to 400 mg/kg;
contains 2′-methoxyacetophenone at a level greater than or equal to 5 mg/kg;
contains 2-methoxybenzoic acid at a level greater than or equal to 1 mg/kg;
contains 4-hydroxyphenllactic acid at a level greater than or equal to 1 mg/kg; and
DNA level from manuka pollen is less than Cq36, which is approximately 3 fg/μL.

A test for multifloral Manuka honey, also adopted by the New Zealand Ministry for Primary Industries, is that the honey has the five following characteristics:
contains 3-phenyllactic acid at a level greater than or equal to 20 mg/kg but less than 400 mg/kg;
contains 2′-methoxyacetophenone at a level greater than or equal to 1 mg/kg;
contains 2-methoxybenzoic acid at a level greater than or equal to 1 mg/kg;
contains 4-hydroxyphenllactic acid at a level greater than or equal to 1 mg/kg; and
DNA level from manuka pollen is less than Cq36, which is approximately 3 fg/μL.

When the term “Manuka honey” is used herein, it refers to a honey that passes at least the DNA test (wherein the DNA level from manuka pollen is less than Cq36, which is approximately 3 fg/μL) for multifloral Manuka honey or for monofloral Manuka honey, as set forth in the previous two paragraphs.

Although not wishing to be bound by the following, several hypothetical reactions and mechanisms of action are described as follows. Manuka honey may symbiotically contribute to the function of the chemical compound in the pharmaceutical composition. One particularly interesting finding is that Manuka contains a high content of methylglyoxal, a highly reactive dicarbonyl compound, which is known to be produced in organisms as a side-product of several metabolic pathways, mainly glycolysis. While endogenous methylglyoxal in animals has been attributed to the formation of advanced glycation end products (or AGEs), which are used as biomarkers in aging and in the development of many degenerative diseases, research suggests that methylglyoxal derived from honey, such as Manuka, does not cause an increase in advanced glycation end products in healthy persons. Furthermore, methylglyoxal in Manuka has been shown to have antibacterial activity against E. coli and S. aureus. attributed it by living cells as a sort of “self-defense” mechanism. This may give the potential for methylglyoxal to make the cancer cells more susceptible to the functions of betulinic acid, along with other compounds found in both Manuka honey and Chaga mushroom.

Manuka honey, as well as other honeys, contains high concentrations of fructose and amino acids, among other molecular compounds formed when the honey is produced. Fructose being found in high concentration, is one of a few types of reducing sugars. Many different amino acids are also found in Manuka and other types of honey in varying concentrations. The ability for fructose and amino acids to react together in an induced reaction has been shown. This type of reaction requires certain parameters and would not spontaneously occur outside of these reaction parameters. This type of documented reaction is known as a Maillard reaction and occurs when a condensation reaction is created between a reducing sugar and an amino acid. Both fructose and glucose are capable of functioning as a reducing sugar during a Maillard reaction with amino acids, undergoing a condensation reaction first to form intermediate products known as Amadori and Heyns products which are two examples of several types of intermediate products formed during a Maillard reaction. During the formation of these intermediate products, methylglyoxal is one of the reactive intermediates that assists in the formation of the final product outcomes of a typical Maillard reaction. The formation of a novel product with a fructose-amine-triterpene, or other constituent, lends to a function of interacting with cancer cells potentially through glucose receptors as well as potentially other cell signal receptors. This possible novel product likely retains a function-structure similar to that observed in other glucoside like therapeutics both found in nature and produced in pharmaceutical labs. A hypothesis proposed here is that the possible novel product could have a similar structure-function and therefore be readily consumed by the cancer cell, thus making betulinic acid and other compounds more bioavailable.

The antimicrobial properties of Manuka honey may also play a role in limiting proliferation of microbes that could be inhibitory to the immune system's destruction of the cancer cells. There is limited understanding of how certain microbes can be a catalyst in the synthesis of betulinic acid during the growing phases of wild Chaga mushroom and is also found in conversion attempts from betulin to betulinic acid in various lab work. Thus, the provision of the “raw materials” via the Chaga extract, in addition to the “blueprint” for betulinic acid with the Chaga mushroom extract, may result in the immune system's own microflora assisting or even accelerating this process of synthesizing higher content of betulinic acid and utilizing this process against cancer cells. This process can be hindered by opportunistic microbes, and interestingly some of those microbe species such as members of the Enterbacter family such as H. pylori have been listed as a carcinogen in humans. It is postulated here that the antimicrobial functions of Manuka also help to create a friendly microbial environment that better facilitates the synthesis and reaction of betulinic acid and other compounds. This function may also facilitate the increasing bioactivity of betulinic acid and allow for better utilization of betulinic acid and other compounds by healthy immune cells.

Another property of Manuka honey is its potent anti-biofilm properties, which may be important due to the nature of bio-film creation by hostile microbes. One of the many, not fully understood, actions against bio-films is to reverse genetic mutations in microbes that have become resistant to antimicrobial treatments, which makes them more easily eradicated by Manuka's own antimicrobial function. Bee defensin is prevalent in all honeys, but the Manuka honey displays some unique protective functions that are not peroxidase based. This may be important in that the Manuka honey compounds do not destroy friendly microbes that could be utilized in synthesis actions as well as protecting healthy cells.

Manuka honey has a relatively low pH, about 3.5-4.5, which contrasts with the higher pH of the Chaga extract and reduction. Furthermore, the lower pH of the honey may contribute to the formula's action on cancer cells because it may be less affected by one of the cancer cell's primary defense mechanisms, i.e., the reduction of pH levels to evade immune cell attacks. This also may permit Manuka to inhibit microbial growth, stimulate the bactericidal actions of macrophages in the host's immune system and, in chronic wounds, to reduce protease activity and increase fibroblast activity and oxygenation.

In addition to the earlier functions discussed, recent studies have demonstrated that Manuka honey can exert anti-proliferative effects against cancer cells. These anticancer properties can involve different processes, including inducing apoptosis in cancer cells through the depolarization of the mitochondrial membrane, inhibiting cyclooxygenase-2 by various constituents (like flavonoids), releasing cytotoxic H2O2, and scavenging of reactive oxygen species (or ROSs). The main mechanism by which Manuka exerts its anti-proliferative effect is through the activation of mitochondrial apoptotic pathways, involving the stimulation of the initiator, caspase-9, which determines the activation of the executioner, caspase-3. This last function is also shared by Chaga mushroom. Moreover, it can induce apoptosis via the activation of PARP, the induction of DNA fragmentation and the loss of Bcl-2 expression.

In vivo, Manuka honey has been shown to be effective in decreasing tumor volume and supporting apoptosis of tumor cells in a mouse melanoma model, reducing colonic inflammation in inflammatory bowel disease in rats, restoring lipid peroxidation and improving antioxidant parameters. Studies show Manuka honey has no detrimental effect in relation to advanced glycation end products nor to a change in gut microbiota homeostasis. This is important as it relates to the unknown properties of how methylglyoxal relates to the progression of certain chronic diseases.

### Carrier

The pharmaceutical composition is one or more of the chemical compositions described herein, dissolved, suspended of emulsified in a pharmaceutically-acceptable liquid for oral consumption. Coconut oil or an alternative medium chain triglyceride may be used to carry the formulation and contribute biochemically to its function, particularly where the formulation is applied topically. The medium chain triglyceride adds a protective layer when the formulation is applied to an open skin lesion as well as subdermal tumors. The carrier is preferably a soft solid, that can be applied as a paste, or a liquid, and can preferably dissolve the Chaga extraction/reduction as well as the honey. In some embodiments the carrier/formulation mixture may be sprayed on to the area to be treated. Coconut oil is a white, solid fat that melts at a temperature of approximately 25° C. to make a clear thin liquid oil. Alternative medium chain triglycerides, for example palm oil, may be liquid at room temperature.

### Chaga Extraction Preparation

An extraction of Chaga mushrooms may be made using the following approach. The Chaga mushroom is chopped to increase the surface area exposed to liquid. In some embodiments, the Chaga is chopped so that 90% of the granules have a maximum dimension of less than 7 mm in maximum dimension, preferably less than about 5 mm, more preferably less than about 3 mm and more preferably less than about 2 mm.

The chopped Chaga is then mixed with an extraction solvent. The extraction solvent typically includes a nonaqueous solvent and may be a solvent typically used for food extraction. Examples of nonaqueous solvents include a short chain alcohol such as ethanol, a short chain glycol such as propylene glycol, a short chain acid, such as methanoic acid, ethanoic acid or lactic acid, a short chain ketone such as acetone, or a short chain ester such as ethyl acetate or n-butyl acetate. If a solvent is used that would be toxic to the patient, it can be removed using standard chemical processing means. The extraction solvent may be present in an amount such that the dry volume of chopped Chaga is about one half the volume of the extraction solvent, although more or less may be used.

Water may be included in the extraction solvent, with the ratio of non-aqueous solvent to water in the extraction solvent being between about 5:95 to I 00:0. Preferably the ratio of non-aqueous solvent to water is in the range 95:5 to 60:40, more preferably in the range 90:10 to 70:30. In other words, the Chaga is mixed into a liquid that includes at least one non-aqueous solvent. The extraction solvent dissolves components of the Chaga released from the mushroom into the solvent. The non-aqueous solvent tends to dissolve the more lipophilic components of the Chaga while the water tends to dissolve the less lipophilic components of the Chaga, since the water is commonly more polar than the non-aqueous solvent. It will be appreciated that other non-aqueous solvents may be used.

The dielectric constant, c, of the extraction solvent is preferably less than 50, preferably less than 40 and may even be less than 30. For example, where the liquid solvent is 100% ethanol, the dielectric constant, c ethanol, is approximately 24.5. In a mixture of miscible liquids, the dielectric constant may be calculated by taking a volumetric average of the dielectric constants. For example, in a liquid containing 90% ethanol by volume and 10% water (ε water=80.1) by volume, the dielectric constant of the mixture is given by (0.9×ε ethanol)+(0.1×ε water)=30.2. The dielectric constant of the liquid is an indicator of the polarity of the solvent mixture.

Larger values indicate that the solvent polarity is higher, which may result in the extraction of less lipophilic constituents of the Chaga mushroom. Lower values of dielectric constant indicate that the solvent polarity is lower, in which case the solvent may be more effective at extracting more lipophilic components from the Chaga mushroom. The solvents used may be either protic or nonprotic. A mixture of ethanol and water is a mixture of two protic solvents.

The Chaga/liquid mixture is then left for a predetermined time suitable for components of the mushroom to be extracted. In some embodiments, the mixture is left at room temperature for more than 48 hours, more than a week, preferably more than a month, more preferably more than two months and even more preferably more than three months. The mixture may be covered or sealed to prevent evaporation of the liquid components. The extraction time may be shorter if the mixture is held at an elevated temperature, for example 90° F., 100° F., or higher, for example up to about 160° F. and/or if the mixture is agitated, for example using a magnetic stirrer. The temperature of the mixture is kept below the boiling point of the liquid.

Another approach to forming an extraction is to use an ultrasonic extraction method, in which ultrasound waves are introduced to the Chaga/liquid mixture. Ultrasonic waves, which may be generated by an ultrasonic probe or other suitable ultrasonic generator travel through the liquid creating alternating high-pressure/low-pressure areas, which can result in acoustic cavitation. This, in turn, can result in locally extreme temperatures and pressures, heating/cooling rates, pressure differentials and high shear forces. When cavitation bubbles implode on the surfaces of the Chaga parts, mass transfer from the Chaga parts into the liquid is enhanced. Under ultrasonic extraction, the Chaga/liquid mixture is exposed to ultrasonic waves for a predetermined period of time, typically a few minutes to hours, to transfer components from the Chaga into the liquid. Ultrasonic extraction may take place at room temperature or at elevated temperatures.

The mixture is then filtered to remove the solids. Any suitable method of filtering may be used, depending on how the liquid extraction is to be used. For example, if the liquid extraction is to be taken orally or applied topically, then the presence of some small Chaga particles, typically <1 mm, may be acceptable, and a filter as coarse as a tea strainer may be acceptable. If, on the other hand, the extract is to be sprayed onto the recipient, then particles as large as 1 mm may block the spray equipment and a finer method of filtering, for example using a filter paper may be used. The extraction is preferably stored in an acid-resistant container, for example a glass container.

The Chaga extract typically has a pH level ranging from 4-9. Generally, the pH of the Chaga extract is lower with longer extraction times and with less polar liquids.

### Chaga Reduction Preparation

A reduction of Chaga mushrooms is made by immersing the chopped Chaga in water, at an elevated temperature for a period of time, or cycled through elevated temperatures for a number of times. In one approach, the Chaga parts are exposed to a boiling or greatly elevated temperature. The Chaga mushroom is chopped so that 90% of the granules have a maximum dimension of less than about 75 mm in maximum dimension, preferably less than about 50 mm, more preferably less than about 30 mm and more preferably less than about 20 mm. The Chaga may also be chopped as finely as discussed above with respect to the extraction, e.g., so that 90% of the granules have a maximum dimension of less than 7 mm in maximum dimension, less than about 5 mm, less than about 3 mm and even less than about 2 mm. The Chaga reduction is made using a solvent that contains at least water and, optionally another liquid, although the solvent used in a reduction is more polar than that used in an extraction.

In one approach, the chopped Chaga is placed in a reduction solvent such as water, preferably in a volume ratio of about 1:40 to about I:2. Preferably the chopped Chaga is covered by the reduction solvent. The reduction solvent may be brought to a boil and then allowed to cool down again in a cycle. During the boiling/cooling cycle, the reduction solvent is brought to a strong boil for a time and kept at an elevated temperature for a time before being allowed to cool off. In some embodiments, the reduction solvent is brought to a strong boil for 1-10 minutes, more preferably 4-5 minutes, and then brought to a simmering boil for about 10-60 minutes, preferably 15-45 minutes, more preferably 20-30 minutes, with the remainder of the cycle permitting the water to cool. An exemplary cycle includes heating to a boil, achieving a rolling boiling for about 4-5 minutes, reducing to a simmering boil for about 20-30 minutes, and cooling for the rest of the cycle. The cycle may take, for example, one hour or more.

In another approach to forming a reduction, the chopped Chaga is heated to an elevated, but not boiling, temperature, for example 160° F. or higher, for a period of time, for example 24-96 hours, 3-7 days, or even longer.

The reduction solvent may include a mixture of different liquid solvents. The dielectric constant, e, of the reduction solvent is preferably more than 50, preferably more than 60 and may even be more than 70. For example, where the liquid solvent is 100% water, the dielectric constant, e water, is approximately 80.1. Here, values of dielectric constant are provided as the d.c. dielectric constant. In a mixture of miscible liquids, the dielectric constant may be calculated by taking a volumetric average of the dielectric constants. For example, in a liquid containing 90% water by volume and 10% ethanol by volume, the dielectric constant of the mixture is given by (0.9×ε water)+(0.1×ε ethanol) 74.5. The dielectric constant of the reduction solvent is greater than the dielectric constant of the extraction solvent, for example, by more than 10, more than 20, or even by more than 30. In an example where an extraction solvent is 9:1 parts ethanol to water by volume, the dielectric constant is about 30.2, whereas in in a reduction solvent containing 9:1 parts water to ethanol by volume, the dielectric constant is about 74.5, a difference of about 34.

The mixture is then filtered to remove the solids. Filtering may be performed after allowing the water to cool. Any suitable method of filtering may be used, depending on how the liquid reduction is to be used. For example, if the liquid reduction is to be taken orally or applied topically, then the presence of some small particles, typically <1 mm, may be acceptable. If, on the other hand, the reduction is to be sprayed onto the recipient, then particles as large as 1 mm may block the spray equipment and a finer method of filtering, for example using filter paper, may be used.

In some approaches, the reduction is formed using a mixture of water and some other, nonaqueous, solvent, such as a food grade solvent. For example a nonaqueous solvent may be a short chain alcohol such as ethanol, a short chain glycol such as propylene glycol, a short chain acid, such as methanoic acid, ethanoic acid or lactic acid, a short chain ketone such as acetone, or a short chain ester such as ethyl acetate or n-butyl acetate If a solvent is used that would be toxic to the patient, it can be removed using standard chemical processing means.

### Extraction/Reduction Mixture Preparation

The extraction may be mixed with the reduction to produce an extraction/reduction mixture (ER mixture). The ratios of volumes of the extraction and reduction used to form the ER mixture may cover a wide range, for ex<:lmple from 50:1 (i.e. 10 parts extraction to 1 part reduction) to 1:50 (i.e. 1 part extraction to 50 parts reduction), preferably from 5:1 to 1:20, more preferably 1:1 to 1:10 and even more preferably 1:3 to 1:7. The volume ratio of extraction and reduction in ER mixture may be around 1:5. In some approaches, the extraction is added to the reduction in an acid-resistant container, for example a glass container.

### Proposed Reaction Mechanism

Although not wishing to be bound by the following proposed reaction mechanism theory, the Maillard reaction occurring in the method of preparing the Extraction/Reduction mixture described above most likely induces a condensation reaction. This might start with fructose and proline, an amino acid in high concentration in honey, and the product of that reaction reacts with methylglyoxal/betulinic acid, since methylglyoxal is a precursor to protein glycation. The condensation reaction with betulinic acid/methylglyoxal and then one or more of the fatty acids could also be set in motion during certain phases of the Maillard reaction when some of the intermediates are very reactive. If so, the reaction could be creating a glycoside-like molecule from the fructose/proline/methylglyoxal/betulinic acid with a fatty acid component. If possible, this could help explain the non-polar/polar indications found in the formula as some of the pH tests show various pH levels in the final assembly. This could also explain the fluorescent nature of the formula found in the first in vitro studies, the final measurements had to be done using an alternative to the fluorescence measurements because of absorbance. If the resulting active ingredient has a glycoside quality, and it survived the digestive system intact, it could be attractive to the glucose receptors on cancer cells and promote binding.

Additional theories for this reaction mechanism are that the Maillard reaction is initiated at the temperature range in which the above-described reaction mixture is incubated; the Maillard reaction has the capacity to induce a condensation reaction, especially if fructose is involved, as it reacts with available carbonyl groups; a reaction between fructose and proline is highly likely, and the product of that reaction is commercially available from Toronto Research Chemicals; the reaction could also involve glycine, or other amino acids; the Maillard reaction could protect the molecule from breaking down in the stomach and small intestine, making it possible to reach the cancer cells while intact; the combination of polar and non-polar starting materials reacting might facilitate a spherical structure, a structure that has been thought to be observed in some of lab tests of substances provided herein; if the structure of the molecule created is a micelle-like structure, that could explain the hydrophobic/hydrophilic characteristics observed; a micelle structure could facilitate transport through the bloodstream in or around the small intestine and is feasible given the observed particle size, it seems feasible.

### Method of Producing a Medicinal Formulation

A dietary supplement, or nutraceutical, is a product intended for ingestion that, among other requirements, contains a dietary ingredient intended to supplement the diet. When composed of natural ingredients, the medicinal formulation provided herein is considered to be, and is equivalent to, a dietary supplement, or nutraceutical. Formulations, including medicinal formulations, may be formed using any combination of the products listed above, including the Chaga extraction, the Chaga reduction, the distillate, and the remaining fraction. In addition, the formulation may include a honey such as a Manuka honey, and a carrier, such as coconut oil or other medium chain triglyceride. For example, an oral formula may include the Chaga extraction, the Chaga reduction and the Chaga distillate in a suitable ratio. Typically, the reduction is present in an oral formula at a greater amount than the Chaga extract or the distillate, although this is not a necessary condition. For example, in the formulation, the ratio of reduction to extract may lie in the range of 100:1 to 1:100, preferably in the range 20:1 to 1:10 and more preferably in the range 20:1 to 1:1. The ratio of reduction:remaining fraction may also lie in the range of 100:1 to 1:100, preferably in the range 20:1 to 1:10 and more preferably in the range 20:1 to 1:1. The ratio of the extract:remaining fraction may lie in the range of 100:1 to 1:100, preferably in the range 10:1 to 1:10 and more preferably in the range 5:1 to 1:5. In one particular example, the weight ratio of the Chaga reduction, Chaga extract and distillate is 5:1:1. Other ratios may be used within the range limits discussed.

Additionally, if administered orally, the formulation may also include Manuka honey typically, but not necessarily, in an amount less than the amount of distillate or extract. For example, the weight ratio of Chaga reduction, Chaga extract, distillate and manuka honey in a formulation may be 10:2:2:1. Other ratios may be used. In particular, the weight ratio of reduction:honey may lie in the range 10:0 to 10:10, or in the range 10:0.1 to 10:10, although ratios higher than about 10:2 may change the consistency of the formulation and also become expensive.

The formulation may also include a carrier, such as coconut oil or other medium chain triglyceride typically, but not necessarily, in an amount less than that of the distillate. The weight ratio of reduction:carrier may lie in the range 10:0 to 10:50, or 10:0.1 to 1:50, depending on the method in which the formulation is to be administered. For example, in a liquid formulation to be applied to a horse's feed, the weight ratio of reduction:carrier may be 10:0 to about 10:2. In one example of a liquid formulation, the weight ratio of Chaga reduction, Chaga extract, distillate and coconut oil in the formulation may be 10:2:2:1. Higher amounts of carrier may be used for a more solid formulation, such as a salve. In an example, the reduction:carrier weight ratio may be 10:10. Other ratios may be used.

The formulation may include both Manuka honey and a carrier such as coconut oil or other medium chain triglyceride. The amount of each of the manuka honey and the carrier may be, but is not required to be, less than that of the extract and distillate. For example, the weight ratio of Chaga reduction, Chaga extract, distillate, manuka honey, and coconut oil in a formulation may be 10:2:2:1:1. Other ratios may be used.

An alternative formulation is provided, which is a reaction between the Chaga extract described above with proline, fructose and a fatty acid such as medium chain triglyceride oil or Sunflower lecithin, or other surfactant, for the esterification process of betulinic acid with fructose and proline to create a fructose-amine-triterpene. These are the isolated starting materials originally supplied by the Manuka honey. The fully synthesized product is suspended in the original formulation, and all material is food grade and FDA approved for sale in original form.

### Administration of Medicinal Formulation

The medicinal formulation may be administered in any suitable manner known to those skilled in the art. The medicinal formulation is a liquid and may be administered orally or applied to solid food. For example, the formulation may be orally consumed by the patient or with the formulation may contain additives, such as sugar, salt, etc., that may be used to alter the flavor to a taste preferred by the patient. In other approaches, the formulation may be added to another liquid to be drunk by the patient. In an animal patient, such as an equine patient, the medicinal formulation may be added to the animal's water. In an animal patient, such as a canine patient, the medicinal formulation may be administered directly into the mouth (per os or PO). In other approaches, the formulation may be mixed together with food. For example, in an animal patient, such as an equine patient, the formulation may be fed directly or may be sprinkled over the horse's feed. When fed directly, an additive may be included to mask the taste of the formulation. One example of a taste-masking agent is sugar. The formulation is well absorbed in pelleted feed. Whole grain feeds and hay, on the other hand do not absorb the formulation as well, in which case the formulation may include a coating agent such as medium chain triglyceride oil so that the formulation adheres to the feed.

In other approaches, the formulation may be applied topically. In some cases, the formulation may be made more viscous when applied topically, for example by adding a more viscous carrier such as coconut or other medium chain triglyceride oil, or a thicker oil. The formulation may be applied topically using a towel, piece of cloth, or pad soaked in the formulation. Another approach is to apply a pad, poultice or the like, which has been soaked in the formulation, to the area to be treated and to hold the pad, poultice or the like in place against the skin. The pad, poultice or the like may be held in place using, for example, adhesive strips, a bandage or any other suitable method. Where the formulation includes a relatively large fraction of carrier, the formulation may have the consistency of a salve that can be spread on the area of concern.

In other approaches, the formulation may be included in a gel, paste or lotion that may be applied to the area to be treated.

In other approaches, the formulation may be sprayed on the area to be treated. In such a case, it may be preferred to include the formulation with a viscous carrier, and for the formulation to be more finely filtered than needed for oral administration, in order to prevent clogging the spraying equipment.

Additional methods of administering the medicinal formulation are provided below with respect to administration of the pharmaceutical compositions containing the isolated or synthesized chemical compound represented by Formula 1 and Formula 2.

The medicinal formulation may be administered to a subject at suitable dose levels. For example, an oral formulation may be dosed daily at between 0.5 ml/10 lbs to 3.0 ml/10 lbs of body weight of the animal patient. The medicinal formulation may be provided in a single daily dose or in two or more smaller doses in a day.

The medicinal formulation may be administered with other therapies. For example, a patient taking the medicinal formulation as described herein may also be on a standard antibiotic regimen.

### Neoplastic Diseases to be Treated

The types of neoplastic disease, or cancer, to be treated by administration of the pharmaceutical composition described here include, but are not limited to, the following cancers: mastocytoma, hemangiosarcoma, splenic hemangiosarcoma, lymphoma, lymphosarcoma, melanoma, osteosarcoma (bone cancer), breast cancer, carcinoma, fibrosarcoma, bladder cancer, brain tumors, oral melanoma, sarcoids, sarcoma, mammary gland carcinoma, oral cancer, transitional cell carcinoma, squamous cell carcinoma, adenocarcinoma, anal gland carcinoma, histiocytic cell tumors, nasal tumors, canine lymphoma, leukemia, and liver cancer. Cancers of epithelial/endothelial (connective tissue), nervous tissue and osteo origin are included.

It will be understood by those skilled in the art that neoplastic disease is a general term for abnormal tissue growth, while cancer is a specific type of neoplasm.

In one embodiment, the cancer to be treated is melanoma, carcinoma, sarcoids, prostate cancer, colorectal cancer, breast cancer, liver cancer and brain cancer.

In another embodiment, the carcinoma to be treated is squamous cell carcinoma, basal cell carcinoma, or Merkel-cell carcinoma.

### Chemical Compounds and Methods of Production

The following compounds can be isolated from the Medicinal Formulation described above and successfully demonstrate anti-cancer activity in vitro.

In one embodiment, the chemical compound is represented by Formula 1 as follows:

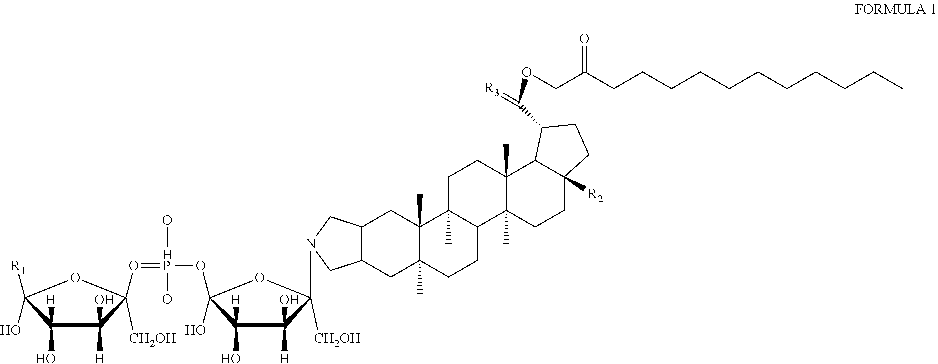

wherein R₁-R₃ are, independently, alkyl/alkane, alkene/alkenyl, or alkyne/alkynyl, having from 1 to 20 carbon atom; benzene/aromatic/phenyl, ether, amide, alkyl halide, amine (-amino), alcohol/hydroxy/hydroxyl (—OH), thiol, aldehyde, ketone, ester, carboxylic acid (COOH), acid anhydride, acyl halide, or methyl; or wherein at least one of R₁, R₂ and R₃ is a hydroxyl group; or wherein R₁-R₃ are hydroxyl.

In another embodiment, the chemical compound is represented by Formula 2 as follows.

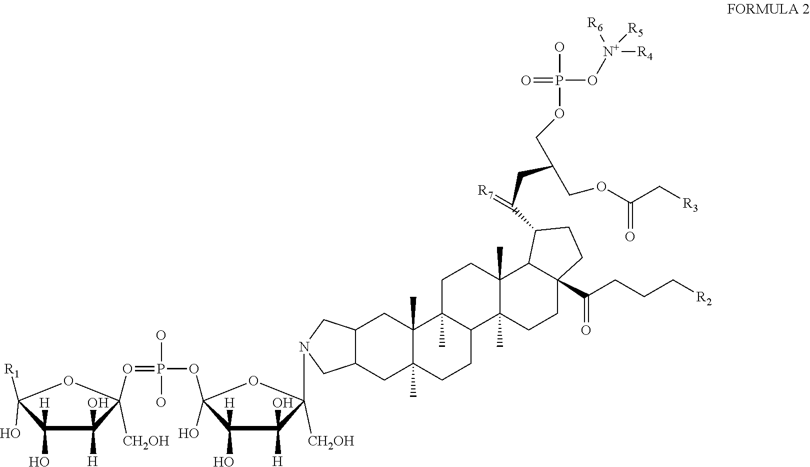

wherein R₁-R₇ are, independently, alkyl/alkane, alkene/alkenyl, or alkyne/alkynyl, having from 1 to 20 carbon atom; benzene/aromatic/phenyl, ether, amide, alkyl halide, amine (-amino), alcohol/hydroxy/hydroxyl (—OH), thiol, aldehyde, ketone, ester, carboxylic acid (COOH), acid anhydride, acyl halide, or methyl; or wherein R₁-R₇ are, independently, alkyl/alkane, alkene/alkenyl, or alkyne/alkynyl, having from 1 to 20 carbon atom; benzene/aromatic/phenyl, ether, amide, alkyl halide, amine (-amino), alcohol/hydroxy/hydroxyl (—OH), thiol, aldehyde, ketone, ester, carboxylic acid (COOH), acid anhydride, acyl halide, or methyl, and wherein at least one of R₁-R₇ is a hydroxyl group; or wherein R₁-R₇ are hydroxyl groups.

### Compositions and Methods of Production

The composition provided herein contains the Medicinal Formulation described above in combination with a suitable carrier. The composition is produced by combining or mixing the Medicinal Formulation provided above with a carrier in accordance with methods known to those skilled in the art.

Suitable carriers include artificial and biological delivery systems such as, but not limited to, liquids, gels, suspensions, and emulsions. Suitable carriers include artificial and biological delivery systems such as, but not limited to, dendrimers, quantum dots, hydrogels, aerogels, foams and creams. Suitable carriers also include nano drug delivery carriers such as, but not limited to, nanospheres, hydrogels with and without nanoparticles, nanocapsules, nanotubes, and nanoparticles. Microsystems are also suitable carriers such as, but not limited to, patches and micropumps. Vesicles of biological or inert origin are also included in the list of suitable carriers, such as, but not limited to, liposomes, aquasomes, niosomes, ethosomes, polymersomes and cubosomes. In addition, carriers having a biological origin such that they provide a class of macromolecules including, but are not limited to, lipids, carbohydrate structures, proteins, peptides and nucleic acids.

Liquids for the formation of solutions, suspensions and emulsions that are particularly suitable for oral delivery to animals, such as mammalian animals, include water, bone broth, animal species-specific herbal teas, and diluted fruit juices. Because the chemical compounds contained in the composition are palatable to animals, the taste of the compounds themselves need not be disguised with natural or artificial flavors or sweeteners.

### Administration of Pharmaceutical Compositions

The pharmaceutical composition may be administered in any suitable manner known to those skilled in the art. Suitable delivery systems include passive delivery systems, such drug delivery via diffusion, or active delivery systems, such as drug delivery via digestion. The pharmaceutical composition may be administered orally, in liquid form or in the form of a solid, such as a pill, capsule, powder or the liquid, suspension or emulsion is applied to animal feed, treats or chewables or squirted directly into the oral cavity using a delivery device such as a syringe; intravenously, topically, subcutaneously, vaginally or rectally, such as with a medicated suppository; via ocular means, such as eye drops, ointments or medicated contact lenses; via transdermal means, such as a patch, via pulmonary means, such as an inhaler, via microelectrochemical systems, or via micropumps. With the potential for nanoparticle carriers, numerous options for administration exist and are known to those skilled in the art.

Oral formulations of the composition may be administered to a patient at suitable dose levels calculated to achieve the desired therapeutic effect. In one embodiment, an oral formulation having 0.5 ml/10 lbs. of body weight to 3.0 ml./10 lbs. of body weight is administered to the mammalian animal to be treated for the dermatologic, neurologic and neoplastic disease or disorder on a daily basis for a sufficient amount of time until disease amelioration or improvement is achieved or maintained. Generally, 30-60 days are sufficient to observe amelioration of symptoms. However, treatment may continue for an indeterminant amount of time to maintain a symptom-free state, particularly when treating a dermatologic disease or condition.

In another embodiment, such as for companion animals particularly canine patients, an oral formulation having a concentration of 0.5 mL/10 pounds of body weight of the Medicinal Formulation in an aqueous solution may be dosed every one to three days. Smaller canine patients may benefit from the higher end of this rate of administration, such as administration every day. Medium and larger canine patients may benefit from administration every two days. And severely diseased canine patients may benefit from a lower frequency of administration, such as at a rate of administration of every three days, and further benefit from a period of rest, or day/days devoid of chemotherapeutic administration (rest day/days), after the first few weeks of administration, until the tumor size has been reduced greater than 50%.

The pharmaceutical composition may be administered with other therapies. For example, the pharmaceutical composition may be administered to a patient simultaneously or sequentially with a second therapeutic agent, such as, but not limited to, a chemotherapeutic drug or a steroid, antibiotic, vitamin, antibody therapy, gene therapy or the like.

The dosing concentrations to be utilized for various small, companion, mammalian animals, such as canines, may be determined as follows. Emesis and diarrhea are to be avoided, therefore 2-3 drops, delivered by conventional eyedropper, of the pharmaceutical composition, delivered as a liquid, suspension or emulsion, are orally administered to the animal, and the animal is observed. When signs of a sleep cycle that is prolonged and deep are observed, this is an indication that the dose is in the preferred therapeutic range for the particular animal undergoing treatment. If no signs of a prolonged and deeper sleep cycle are observed, the number of drops of pharmaceutical composition is increased until the desired effect is reached. As mentioned above, this concentration dose for canines is an oral formulation having a concentration of 0.5 mL/10 pounds of body weight of the Medicinal Formulation in an aqueous solution, dosed every one to three days.

The invention will now be further described by reference to the following non-limiting examples

### EXAMPLES

### Example 1: Toxicity Study of Pharmaceutical Composition for Treatment of Canine Cancer

Consistent trends in dosing were achieved in real-life scenarios that look to simulate what will be realistic applications of the canine drug therapies provided herein.

This Pilot Study successfully validated the safe dosing range for administration of an aqueous solution of the Medicinal Formulation, observed at 0.5 mL/10 pounds every 24-72 hours, depending on breed, with +/−0.2 ml/10 lbs. safety margin while maintaining efficacy.

No toxicity or adverse reactions were reported.

All subjects began with loading dose of 1-3 drops of a 10 μg/mL concentration of the chemical compound of Formula 1, wherein R1-R3 are hydroxyl, in water, administered orally every 2 to 3 days for 30 days.

### Oral Liquid Suspension

The safety of a pharmaceutical composition containing an aqueous solution of the Medicinal Formulation containing the chemical compound of Formula 1, wherein R1-R3 are hydroxyl, at a concentration of 10 μg/mL was previously elucidated in other small population surveys. Given multiple variables of diagnosis variety, dog age/breed and limitations of owner assessment outside of blood work and clinical assessment from clinicians, the finding of consistent trends of improvement/complete resolution of disease in all cases that were able to complete the study is significant and validates the legitimate need for further study.

### Outcome of the Study

Seven of the nine dogs enrolled were able to complete the study. One of the nine dogs was euthanized prior to commencing treatment and another one of the nine dogs was euthanized without confirmation of treatment protocol followed by owner. Of the seven dogs that completed treatment, all seven showed significant improvement with treatment ongoing beyond the timeline of this study or complete resolution of disease. Clinical assessments for visual changes, scans and blood chemical panels were used to assess all patients undergoing treatment in the study. In addition to measured outcomes, all owners participating in the pilot study reported additional benefits in their dog's condition including improved mobility, significant increases in energy, improved eyesight and general quality of life. All seven dogs continued to consume the pharmaceutical composition as a daily supplement for overall quality of life or further treatment to prevent recurrence. The continuation of research to better ascertain additional and long-term applications for the pharmaceutical composition in dogs would be of tremendous value to the veterinary community as a whole.

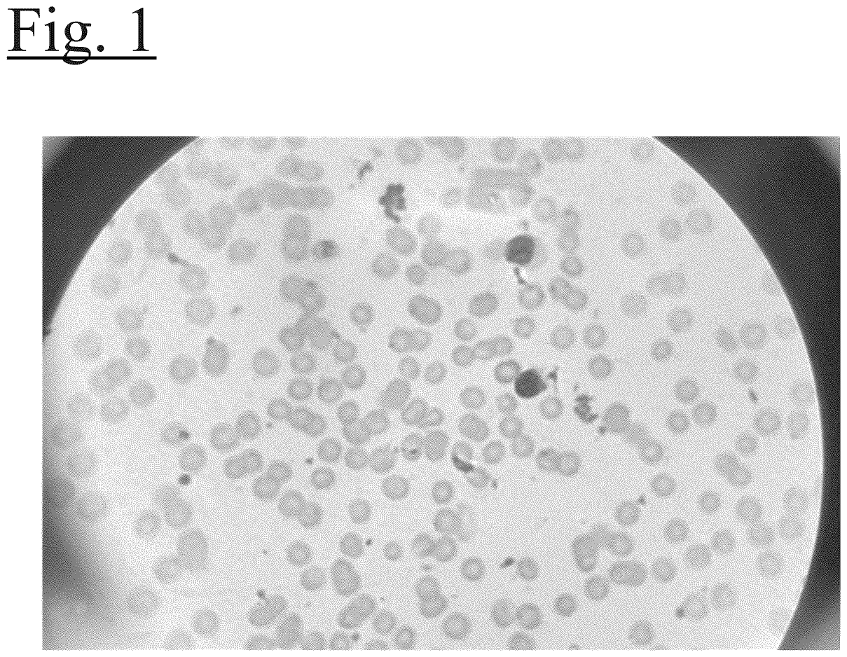

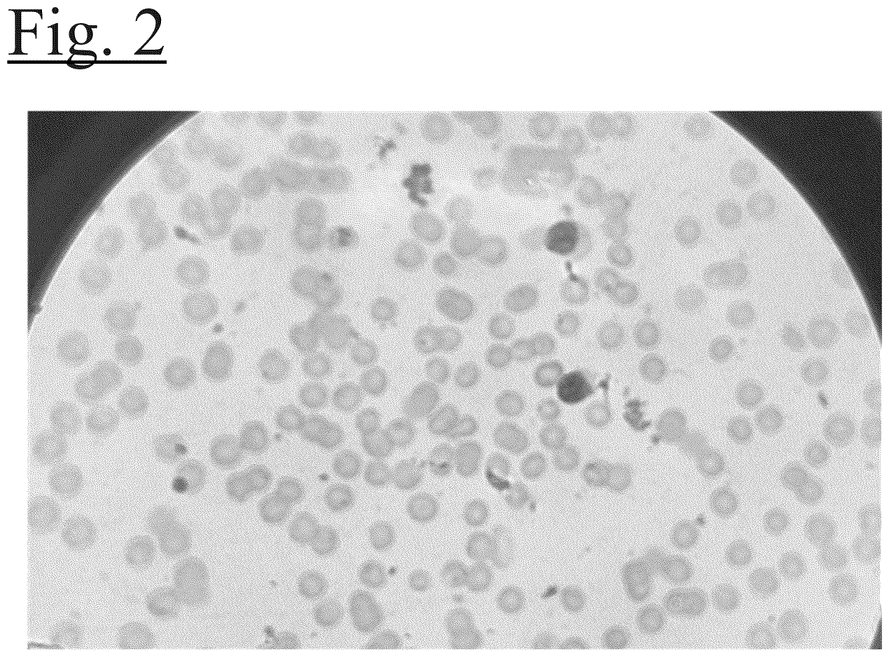

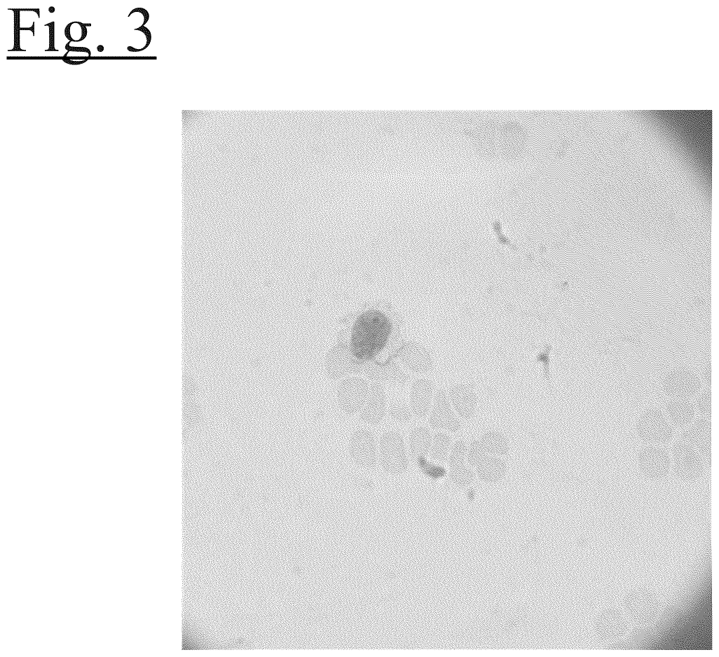

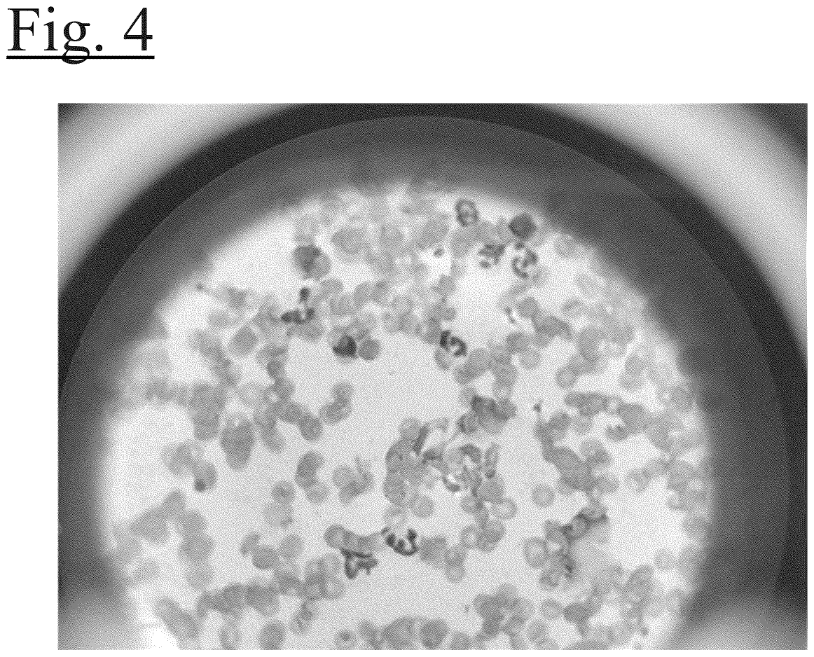

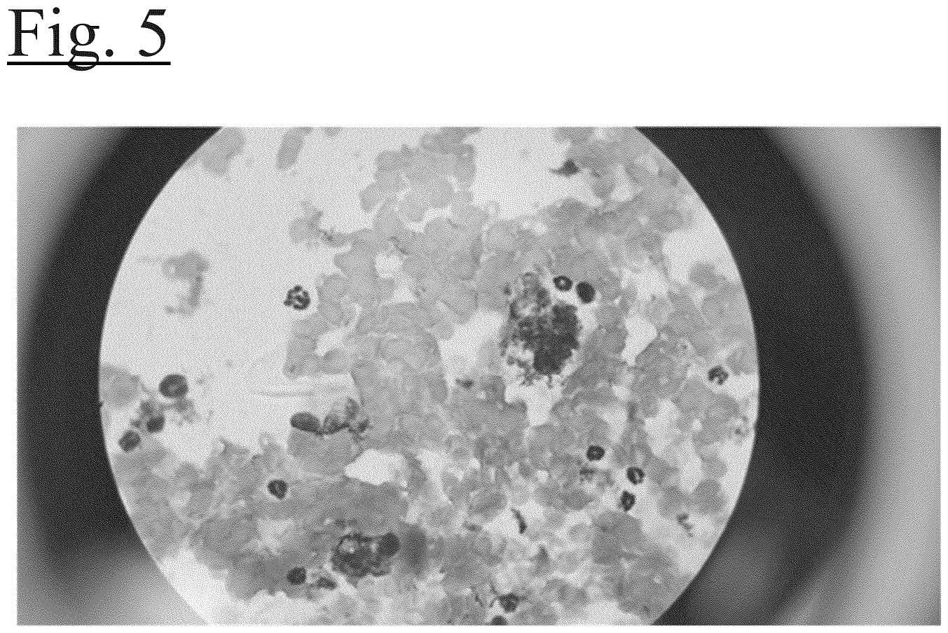

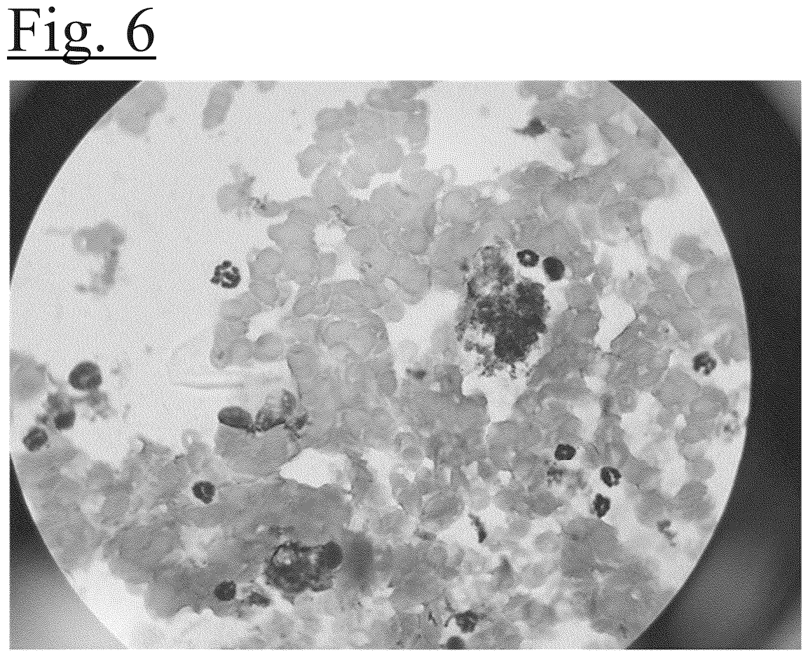

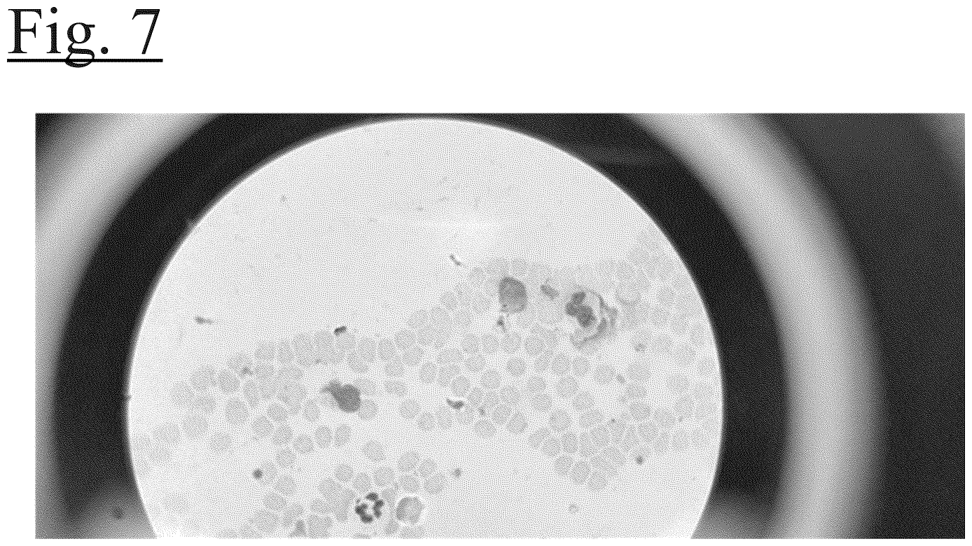

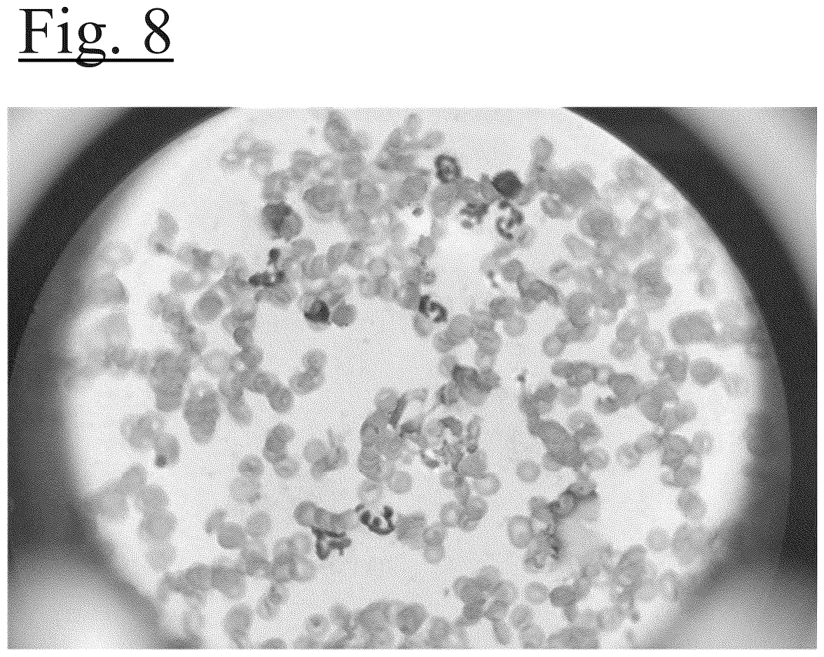

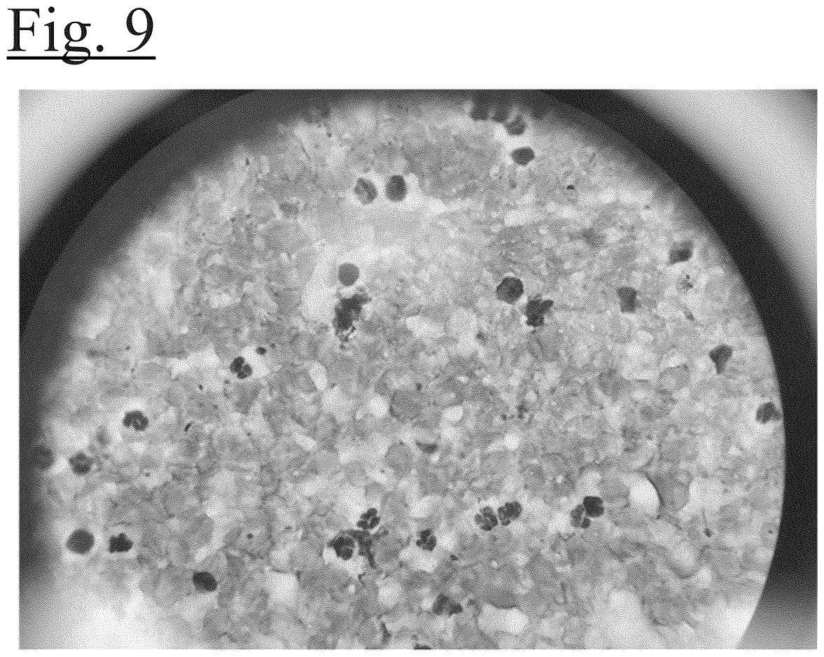

The images in FIGS. 1-9 show immune cell activity in tumor biopsy of patients in this canine study.

Diff-quick stain was used to identify neutrophils, monocytes, macrophages and lymphocytes.

Cancer cells show hallmark signs of plasma membrane inversion indicating apoptosis has begun. The cell membrane inversion is important for the contents of the cell to be contained during death, not triggering an over-reactive inflammatory response.

Some images provided in FIGS. 1-9 show active immune cell (T-cell) destruction and phagocytosis of malignant cells.

The images in FIGS. 1-9 also show malignant cells displaying signs of beginning apoptosis. Membrane blebbing, external particulates and cell fragments are minimal, most likely indicting correct cell death cycles. Malignant tissue lacking contact inhibition was easily identified in several images. Immune cell adhesion to malignant tissue in addition to phagocytosis was well demonstrated.

Notable is the presence of cell membrane inversion. It may be seen and understood that cells are not becoming necrotic, which is really important for overall health and not triggering an overaggressive immune response and not causing an overreactive inflammatory response that can cause pain and stress on the body. The fact that the cell membranes invert and the contents are contained means the cells die and are then ready for consumption and recycling, which is advantageous.

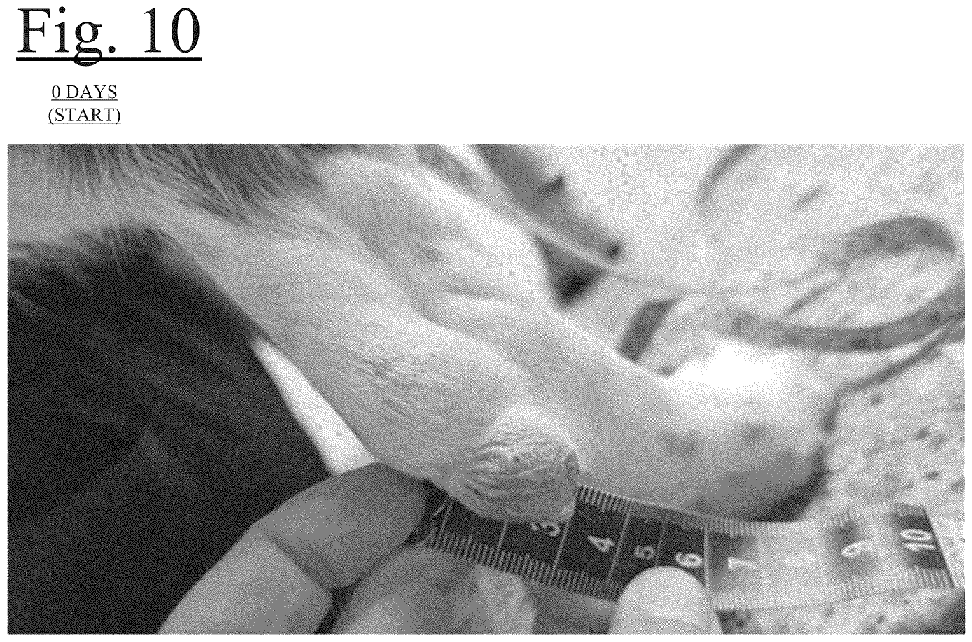

FIG. 10 is a photographic image of the leg of a nine-year-old beagle with a malignant tumor on the right hind leg immediately prior to treatment.

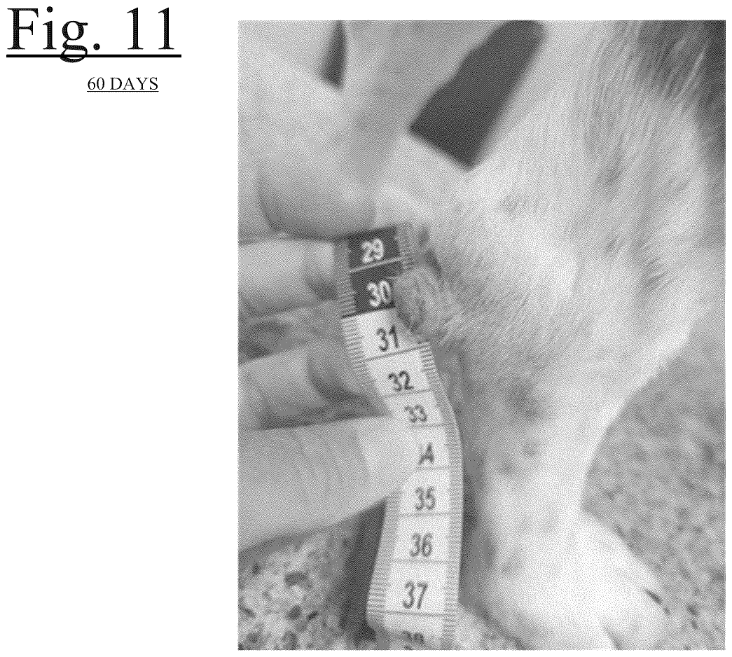

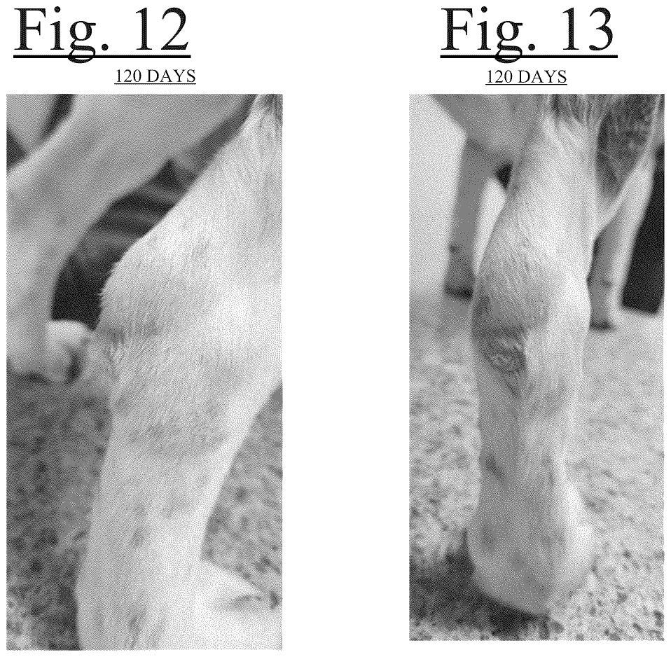

The images in FIGS. 11-13 show the gradual decrease of tumor size and necrosis.

### Example 2: Safety and Efficacy Study of Medicinal Formulation

This study was designed to evaluate the safety and tolerability of the medicinal formulation described herein and commercially available as the nutraceutical, Mytopaws™ Essential (Mytosynth Nutraceuticals, Bloomington, MN).

### Study Design:

Type: Single treatment arm
Duration: Twelve weeks
Participants: Minimum of thirty volunteers, maximum of sixty

### Statistical Significance:

Sample Size Calculation: To detect a clinically significant difference in the primary endpoint with 80% power and a significance level of 0.05, assuming a moderate effect size, a minimum of 50 participants per group is required.

### Inclusion Criteria:

1. Any malignancy that has been definitively diagnosed with a biopsy by a boarded pathologist when the owner declines conventional treatment
2. Any mass highly suspicious for malignancy which is seen on physical exam and/or on imaging AND for which the owner declines biopsy or a biopsy is not recommended because of the invasive or risky nature of the biopsy procedure. (For example, lung or splenic tumors where the risk of the biopsy procedure is substantial)
3. Ability to comply with study protocol and procedures

### Exclusion Criteria:

1. Known hypersensitivity to any component of the supplement

### Key Laboratory and Exam Tests:

1. CBC and electrolyte panels
2. Liver Function Tests (Alkaline phosphatase, ALT, AST)
3. Renal Function Tests (Creatinine, BUN)
4. Ultrasound, x-ray, CT scan and/or MRI if available
5. Tumor measurements when available
6. Weight—Scales checked daily with a weight standard. Canines to be checked at baseline, four weeks, eight weeks and twelve weeks

### Results:

No toxic effects were observed.

### Example 3: Quality of Life (QoL) Study to Evaluate Neurologic Disease Treatment

This study is designed to evaluate effects on the Quality of Life of an animal subsequent to administration of the medicinal formulation described herein, which is commercially available as the nutraceutical, Mytopaws™ Essential (Mytosynth Nutraceuticals, Bloomington, MN). A Quality of Life assessment provides an indication of neurologic disease progression or amelioration.

### Study Design:

Type: Single treatment arm.
Duration: Twelve weeks.
Participants: Minimum of thirty volunteers, maximum of sixty.

### Statistical Significance:

Sample Size Calculation: To detect a clinically significant difference in the primary endpoint with 80% power and a significance level of 0.05, assuming a moderate effect size, a minimum of fifty participants is required.

### Inclusion Criteria:

1. Age—Canines 9 years and older.
2. Stable medication regimen for at least 3 months prior to study entry.
3. Maladies—Looking for common, age and non-age-related maladies that have symptoms that are easily measurable and whose improvement would also be easily measurable.
   a) Weight Issues—Usually overweight, however many geriatric pets are losing muscle and weight. Purina has a well-accepted body condition score. It is included in the packet for reference.
   b) Arthritis—Pets whose owners present with a complaint of weakness, difficulty rising, difficulty walking or complaints of pain WITH specific symptoms such as carrying a limb or licking a joint. Pets can also have a history of orthopedic injury that was surgically repaired, treated with medication, or was never treated. (Cruciate injury for example).
   c) Pets will have one or more of the following.
      i. A measurable gait abnormality with a force plate or by visual evaluation and video evidence.
      ii. Radiographic evidence of OA or other significant abnormality.
      iii. Palpable changes on physical exam such as loss of muscle mass, buttressing of stifles or elbows, joint effusion, limited range of motion, or boney callus.
      iv. Specifically EXCLUDED are pets with acute injury or intervertebral disk disease.
4. Healthy Pets—Normal weight pets with no evidence of OA.
5. Ability to comply with study protocol and procedures.

### Exclusion Criteria:

1. Participation in another clinical trial within the last 3 months.
2. Known hypersensitivity to any component of the supplement.

### Key Laboratory Tests:

1. CBC and electrolyte panels.
2. Liver Function Tests (Alkaline phosphatase, ALT, AST).
3. Renal Function Tests (Creatinine, BUN).

### Purina Body Condition System Score:

1. See attached.

### Quality of Life Questionnaire:

Validated Tool: Adapted from an Ohio State Quality of Life Owner Survey
   Administered online or in person at the beginning of the study, at six weeks and at twelve weeks.

| | |
|---|---|
| Has Difficulty Rising. | Score (1-10): |
| No longer wants to take or complete their normal walks. | Score (1-10): |
| Is lame in one or more legs. | Score (1-10): |
| Takes medication every day to alleviate pain or lameness. | Score (1-10): |
| Has difficulty squatting to urinate or defecate. | Score (1-10): |
| Has lost their appetite. | Score (1-10): |
| Has diarrhea regularly. | Score (1-10): |
| Vomits at least once a week. | Score (1-10): |
| Is losing weight and muscle mass. | Score (1-10): |
| Is not as interactive with me or other members of the family. | Score (1-10): |
| Is urinating or defecating in the house. | Score (1-10): |
| Trembles or shakes at times. | Score (1-10): |
| Sleeps more than normal. | Score (1-10): |
| Does NOT seem to enjoy life. | Score (1-10): |
| Seems mentally dull or depressed. | Score (1-10): |
| Does NOT want to play. | Score (1-10): |
| Aimless wandering, unable to settle, pacing. | Score (1-10): |
| Increased anxiety when separated from owners, | Score (1-10): |
| leaving home, or increased noise reaction. | |
| More irritable or aggressive. | Score (1-10): |
| Total Score: | Rate Your Pets Overall Health |
| | (Note 1 = Excellent to 10 = Poor): |

### Objective Measurements:

1) Weight—Scales checked daily with a weight standard. Canines to be checked at baseline, six weeks, and twelve weeks.
2) Pit Pat Activity Monitors—Sleep, exercise, calorie burn, and distance traveled.
3) Orthopedic exam including range of motion at baseline, six weeks, and twelve weeks.

### Study Procedures:

### 1. Screening Phase (Week −4 to 0):

Select candidates for study inclusion.
Screen for eligibility (medical history, physical examination, x-ray, and/or lab tests as needed).

### 2. Baseline Visit (Week 0-1):

Collect baseline data (weight, lab tests, exam findings, owner questionnaire).
Dispense study product (Mytopaws) to include dosage (see below) and study instructions/information. Be certain canines have Pit Pat Activity Monitor on for two weeks prior to administration of Mytopaws to obtain baseline activity level.

### 3. Treatment Phase (Week 1-12):

Canines take the study product daily with first meal of the day and document with a photo or a text.
Owner questionnaire, canine physical exam and blood work at six weeks and twelve weeks.
Collect adverse events and compliance data.

### 4. End of Study Visit (Week 12):

Final lab tests and physical examination.
Review and collect unused study product.

### Data Analysis:

Primary Endpoint: Change in QoL scores.
Secondary Endpoints: Change from baseline blood values, weight, activity levels, and/or physical exam findings.
Paired t-tests or Wilcoxon signed-rank tests: (Depending on data distribution) will be used to assess changes in baseline and end-of-study scores, findings and values.

### Safety Monitoring:

Regular monitoring of adverse events (can be done by phone/email).
Interim safety analysis at midpoint of study.
Immediate reporting of serious adverse events to the principal investigator.

### Conclusion:

This protocol aims to rigorously assess the effectiveness and safety of Mytopaws in managing improved health and wellness, contributing to evidence-based recommendations for its use in clinical practice.

### Dosage Instructions:

Start at 0.1 mL/10 lbs. for 2 days, advance to 0.2 mL/10 lbs. for 2 days. If tolerated (i.e. no new or worsening diarrhea, vomiting, or other significant changes) advance to 0.5 mL/10 lbs. for the remainder of the study. The product can be given orally or added to food.

0.5 mL <10 pounds
1.0 mL 10-20 pounds
1.5 mL 20-30 pounds
2.0 mL 30-40 pounds
2.5 mL 40-50 pounds
3.0 mL 50-60 pounds
3.5 mL 60-70 pounds
4.0 mL 70-80 pounds
4.5 mL 80-90 pounds
5.0 mL 90-100 pounds
5.5 mL 100-110 pounds
6.0 mL 110 pounds

For dogs over 110 pounds continue to increase dose as needed to keep with the 0.5 mL/10 lbs. schedule.

### Results:

Pet owners reported that an improved quality of life was observed in dogs that received the medicinal formulation. Quantifiable data is presented in FIG. 17, which is a line graph of neurologic symptoms for each of 7 dogs to whom have been administered the Medicinal Formulation. A Symptom Score of 10 indicates extremely bad neurologic symptoms, whereas a Symptom Score of 0 indicates that the symptoms have completely resolved. The line marked “A” represents the neurologic symptom scores for each dog on Day 0 of treatment, which was oral administration of the Medicinal Formulation. The line marked “B” represents the neurologic symptom score for each dog on Day 60 of treatment. And the line marked “C” represents the neurologic symptom score for each dog on Day 90 of treatment. For example, Dog 1 had a Symptom Score of 4 on Day 1, a Symptom Score of 0 on Day 60 of treatment and also a Symptom Score of 1 on Day 90 of treatment.

### Example 4: Efficacy Study for Treatment of Dermatologic Disease

This study is designed to evaluate the efficacy of the Medicinal Formulation described herein, which is commercially available as the nutraceutical, Mytopaws™ Essential (Mytosynth Nutraceuticals, Bloomington, MN).

### Objectives:

Primary Objective: To evaluate the efficacy of improvements in skin lesions, itch score, organism counts and the need for additional therapeutics to control clinical signs and symptoms.
Secondary Objective: To assess the safety and tolerability of Mytopaws.

### Study Design:

Type: Single treatment arm.
Duration: Twelve weeks.
Participants: Minimum of thirty volunteers, maximum of sixty.

### Statistical Significance:

Sample Size Calculation: To detect a clinically significant difference in the primary endpoint with 80% power and a significance level of 0.05, assuming a moderate effect size, a minimum of fifty participants is required.

### Inclusion Criteria:

1. Any pet that presents with complaints of itching, skin rash, pinnae dermatitis, alopecia, or other skin lesion AND is diagnosed with Atopic Dermatitis, Flea Allergic Dermatitis or Food Allergy.
2. Ability to comply with study protocol and procedures.

### Exclusion Criteria:

1. Known hypersensitivity to any component of the supplement.
2. Pets with severe allergic disease where traditional parental or oral therapy is required, will be excluded. These would include Cyclosporine, corticosteroids, Cytopoint and Apoquel. Use of topical therapies including shampoos, lotions and ear medications will be allowed.

### Key Laboratory and Exam Tests:

1. CBC and electrolyte panels.
2. Chemistry Panel to include at a minimum: glucose, albumin, total protein, BUN, Cr, ALT, and ALP.
3. Pruritis Visual Analog Scale (PVAS) taken from the Canadian Academy of Veterinary Dermatology. Benefits—simple for the owner to understand and report as a single number. Validated for use in clinical studies. See attached.
4. Skin Diagnostics including ear cytology, skin scrapings, cytology, and skin impressions. Organisms identified including yeast, cocci bacteria and rod bacteria to be noted and scored on a scale of 0 (absent) to 4 (abundant).

### Study Procedures:

### 1. Screening Phase (Week −4 to 0):

Select candidates for study inclusion.
Screen for eligibility (medical history, diagnostic—skin scrapings, skin impressions, cytology, and ear cytology as indicated.)

### 2. Baseline Visit (Week 0-1):

Collect baseline data (weight, test results, owner completes the PVAS questionnaire).
Take photograph(s) of the skin lesions. Measure the skin lesions.
Baseline diagnostics including skin scrapings, skin impressions, cultures, cytology and ear cytology were performed.
Dispense study product (Mytopaws) to include dosage (see below) and study instructions/information.
Patients will receive appropriate topical therapy such as ear medications and shampoo however we will avoid parental and oral medications including corticosteroids, Cyclosporine, Cytopoint and Apoquel. If patients have severe disease where these treatments are deemed necessary, they will be excluded. Pets will be allowed to take flea treatment as the pruritis from flea allergy can be expected to last after the last flea is gone which may necessitate repeated flea treatments. It will be important to know if Mytopaws is a treatment for the tailhead alopecia and pruritis caused by flea allergy. Flea allergic pets are often treated with flea killing medication and an anti-itch medication such as a corticosteroid.

### 3. Treatment Phase (Week 1-12):

Canines take the study product daily with first meal of the day and document with a photo or text.
Owner questionnaire, canine physical exam, repeat diagnostics as indicated and blood work at four weeks, eight weeks and twelve weeks.
Collect adverse events and compliance data.

### 4. End of Study Visit (Week 12):

Final lab tests, final diagnostics and physical examination.
Review and collect unused study product.

### Data Analysis:

Primary Endpoint: Decrease in pruritis score, skin lesions, and reduction in traditional therapies required to keep pet comfortable.
Secondary Endpoints: Change from baseline blood values.
Tertiary Endpoint: Pets whose clinical signs do not improve or intensify and require additional therapy with approved parental or oral medications.
Paired t-tests or Wilcoxon signed-rank tests: (Depending on data distribution) will be used to assess changes in baseline and end-of-study scores, findings and values.

### Safety Monitoring:

Regular monitoring of adverse events (can be done by phone/email).
Interim safety analysis at four weeks.
Immediate reporting of serious adverse events to the principal investigator.

### Conclusion:

This protocol aims to rigorously assess the effectiveness and safety of Mytopaws in managing canine allergic disease. Canine allergic diseases include Atopic Dermatitis, Food Allergy Dermatitis and Flea Allergy Dermatitis. Alopecia, skin erythema, papules, pustules, macules, pyoderma, otitis externa and pruritis are characteristic of allergic disease in pets. Many times, diagnosis of the exact allergy is difficult and controlling exposure not practical. Therefore, many pets receive chronic allergy medications which can have substantial side effects and may not be efficacious. Pets who have allergy symptoms that do not require immediate use of approved parental or oral medication will be included. Topical therapies will be allowed in order to provide some relief to pets and to control bacterial dermatitis, yeast dermatitis and bacterial or yeast otitis.

### Dosage Instructions:

Start at 0.1 mL/10 lbs. for 2 days, advance to 0.2 mL/10 lbs. for 2 days. If tolerated (i.e. no new or worsening diarrhea, vomiting, or other significant changes) advance to 0.5 mL/10 lbs. for the remainder of the study. The product can be given orally or added to food.

0.5 mL <10 pounds
1.0 mL 10-20 pounds
1.5 mL 20-30 pounds
2.0 mL 30-40 pounds
2.5 mL 40-50 pounds
3.0 mL 50-60 pounds
3.5 mL 60-70 pounds
4.0 mL 70-80 pounds
4.5 mL 80-90 pounds
5.0 mL 90-100 pounds
5.5 mL 100-110 pounds
6.0 mL 110 pounds

For dogs over 110 pounds continue to increase dose as needed to keep with the 0.5 mL/10 lbs. schedule.

### Dosing Summary for Canine Study

1) Three research arms:
   a) Senior QOL (quality of life)
   b) Oncology
   c) Allergy-combination therapy
2) Senior QOL—55+ dogs
   a) General wellness scores increased ˜90% improvement for mobility/vitality
   b) Dosing regimen: 0.5 mL/10 lbs
3) Oncology −7 dogs
   a) Safety parameters for dosing regimen established at: 0.5 mL/10 lbs up to 1 mL/2 lbs. **Will be increasing to 1 mL/0.5 lbs. 10/25**
   b) Confirmatory trends
4) Allergy—38 dogs
   a) Clinical symptoms improved ˜90% on average both mono/combination therapy
   b) Decrease of side effects from other drugs observed c) Dosing regimen: 1 mL/10 lbs up to 3 mL/10 lbs

### Results:

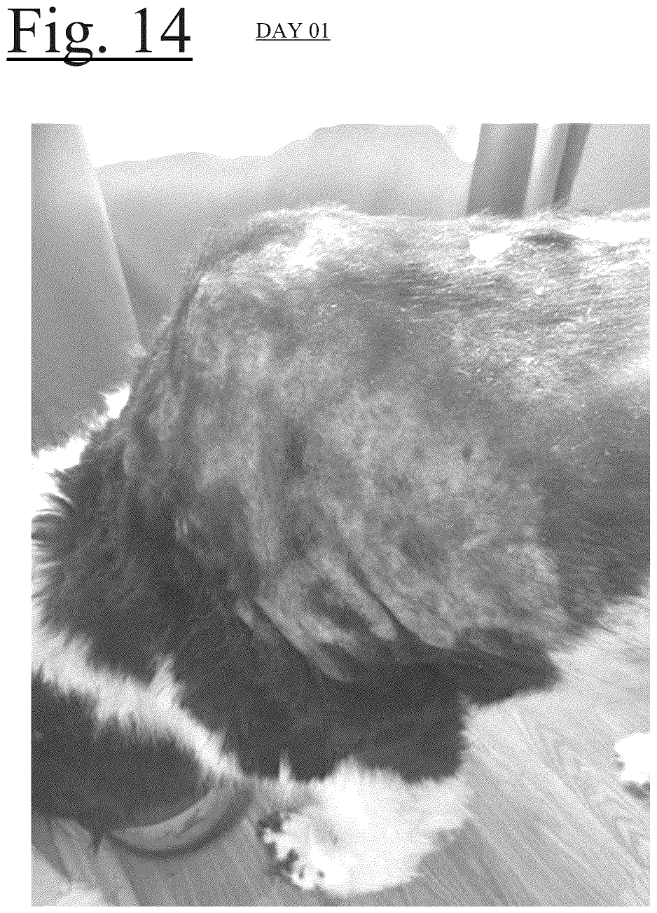

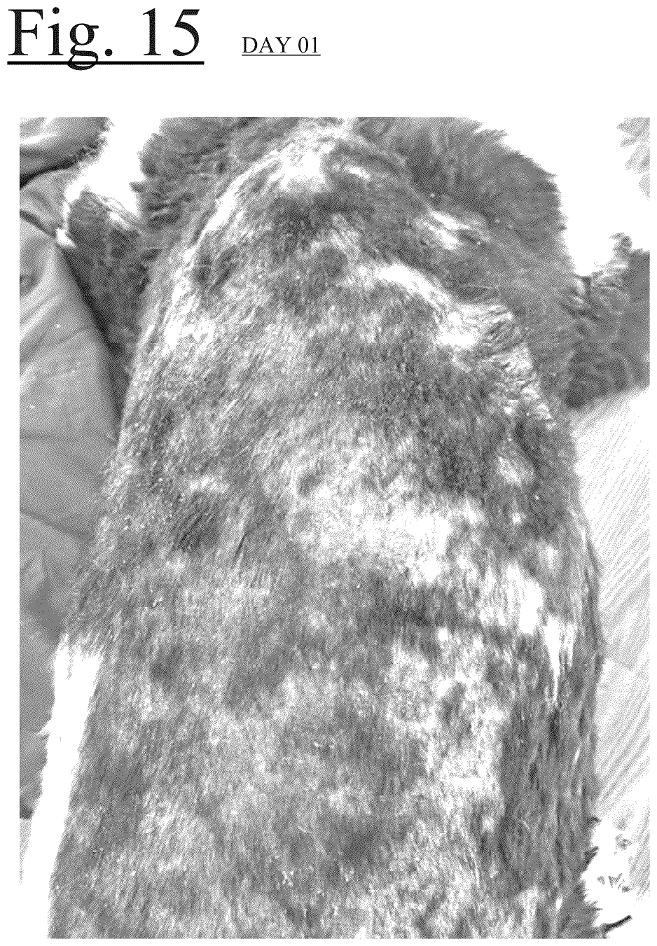

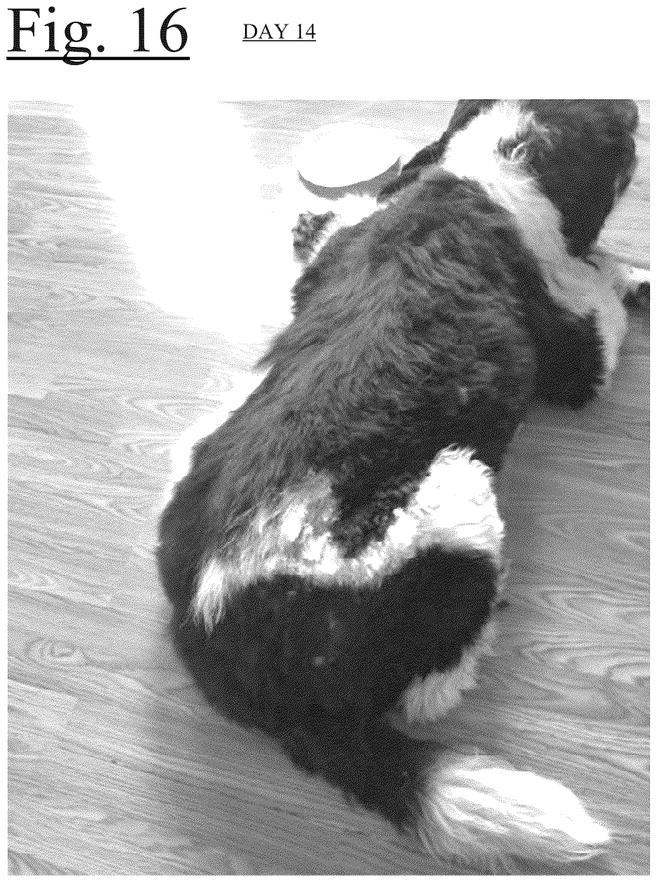

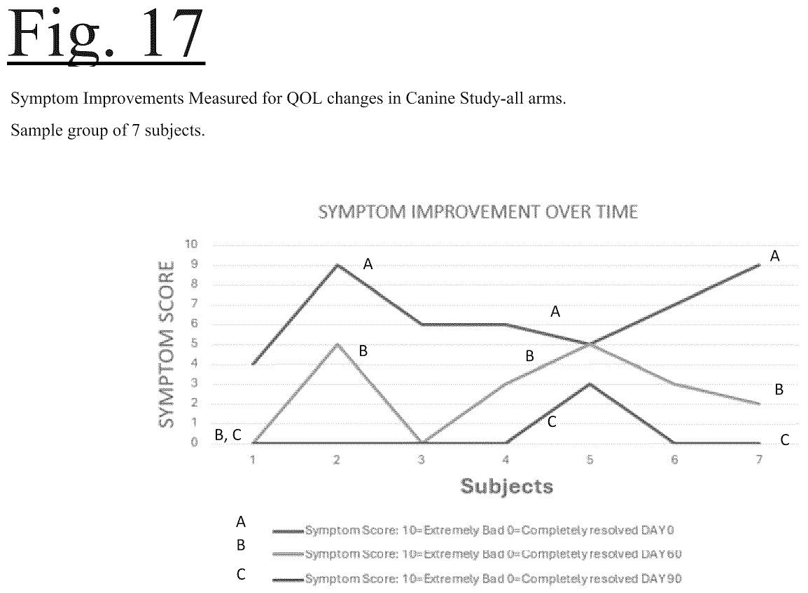

The dogs that received the medicinal formulation were observed to have improved dermatologic disease and allergy symptoms as shown in FIGS. 14-16. FIG. 14 is a photograph of the shoulders and back of the dog on Day 01 of the study. Fur is very thin or absent, leaving bald sections of skin, which is splotchy. FIG. 15 is another photograph of the dog on Day 01 of the study taken from the rear of the dog. Patches of baldness and fur thinning can be seen. FIG. 15 is a photograph of the same dog on Day 14 of treatment from tail to head. Fur has grown in full and luxurious, no bald patches can be seen, tail is full, color of fur is vibrant.

As noted above, the present invention is applicable to pharmaceutical composition containing the chemical compounds described herein and methods of making and using thereof. Accordingly, the present invention should not be considered limited to the examples described above, but rather should be understood to cover all aspects of the invention as fairly set out in the attached claims.

What is claimed is:

1. A method of treating a neoplastic disease in a mammalian subject, comprising administering to the subject an effective amount of a Medicinal Formulation, wherein the Medicinal Formulation is an emulsified Chaga mushroom solution containing chemical-modified compounds of the Chaga mushroom.

2. The method of claim 1, wherein the neoplastic disease is cancer.

3. The method of claim 1, wherein the cancer is melanoma, carcinoma, sarcoids, prostate cancer, colorectal cancer, breast cancer, liver cancer and brain cancer.

4. The method of claim 1, wherein the carcinoma is squamous cell carcinoma, basal cell carcinoma, or Merkel-cell carcinoma.

5. A method of treating a dermatologic disease in a mammalian subject, comprising administering to the subject an effective amount of a Medicinal Formulation, wherein the Medicinal Formulation is an emulsified Chaga mushroom solution containing chemical-modified compounds of the Chaga mushroom.

6. The method of claim 5, wherein the dermatologic disease is atopic dermatitis, flea allergy dermatitis, contact dermatitis, bacterial infections, fungal infections or parasitic infestation.

7. The method of claim 6, wherein the dermatologic disease is a bacterial infection, and the bacterial infection is pyoderma.

8. The method of claim 6, wherein the dermatologic disease is a fungal infection, and the fungal infection is ringworm

9. The method of claim 6, wherein the dermatologic disease is a parasitic infestation and the parasitic infection is selected from the group consisting of mites and scabies.

10. A method of treating a neurologic disease in a mammalian subject, comprising administering to the subject an effective amount of a Medicinal Formulation, wherein the Medicinal Formulation is an emulsified Chaga mushroom solution containing chemical-modified compounds of the Chaga mushroom.

11. The method of claim 10, wherein the neurologic disease is epilepsy, stroke, a brain tumor, a vestibular disease, degenerative myelopathy, an infectious disease, or a trauma.

12. The method of claim 11 wherein the trauma is traumatic brain injury, trauma to the central nervous system, trauma to the peripheral nervous system, drug trauma, traumatic accidental injury, or blunt force trauma.

13. The method of claims 1, 5 or 10 wherein the effective amount is a therapeutic dose of the Medicinal Formulation that treats the disease.

14. The method of claim 13, wherein the therapeutic dose is from 0.5 ml/10 lbs. of body weight to 3.0 ml/10 lbs. of body weight.

15. The method of claims 1, 5 or 10, wherein the administration is oral administration.

16. The method of claim 15, wherein the Medicinal Formulation is combined with a carrier to create a composition that facilitates oral administration.

17. The method of claims 1, 5 or 10, wherein the Medicinal Formulation contains a chemical compound having the formula of Formula 1, wherein R¹, R² and R³ are independently selected from the group consisting of alkyl/alkane, alkene/alkenyl, or alkyne/alkynyl, having from 1 to 20 carbon atom; benzene/aromatic/phenyl, ether, amide, alkyl halide, amine (-amino), alcohol/hydroxy/hydroxyl (—OH), thiol, aldehyde, ketone, ester, carboxylic acid (COOH), acid anhydride, acyl halide, or methyl, in a pharmaceutically-acceptable carrier; or wherein at least one of R¹, R² and R³ is a hydroxyl group; or wherein R¹, R² and R³ are hydroxyl groups

18. The method of claims 1, 5 or 10, wherein the Medicinal Formulation contains a chemical compound having the formula of Formula 2, wherein R₁-R₇ are, independently, alkyl/alkane, alkene/alkenyl, or alkyne/alkynyl, having from 1 to 20 carbon atom; benzene/aromatic/phenyl, ether, amide, alkyl halide, amine (-amino), alcohol/hydroxy/hydroxyl (—OH), thiol, aldehyde, ketone, ester, carboxylic acid (COOH), acid anhydride, acyl halide, or methyl; or wherein R₁-R₇ are, independently, alkyl/alkane, alkene/alkenyl, or alkyne/alkynyl, having from 1 to 20 carbon atom; benzene/aromatic/phenyl, ether, amide, alkyl halide, amine (-amino), alcohol/hydroxy/hydroxyl (—OH), thiol, aldehyde, ketone, ester, carboxylic acid (COOH), acid anhydride, acyl halide, or methyl, and wherein at least one of R₁-R₇ is a hydroxyl group; or wherein R₁-R₇ are hydroxyl groups.
